(19) United States
(12) Patent Application Publication
(10) Pub. No.: US 2026/0294616 A1
(43) Pub. Date: Oct. 1, 2026

(54) CROSSLINKABLE LIQUID COMPOSITION FOR USE IN A METHOD OF TREATING AN EYE DISORDER

(71) Applicants: Universiteit Antwerpen, Antwerpen (BE); Universitair Ziekenhuis Antwerpen, Edegem (BE)

(72) Inventors: Bert VAN DEN BOGERD, Wilrijk (BE); Sorcha NÍ DHUBHGHAILL, Edegem (BE)

(73) Assignees: Universiteit Antwerpen, Antwerpen (BE); Universitair Ziekenhuis Antwerpen, Edegem (BE)

(21) Appl. No.: 19/478,594

(22) PCT Filed: Apr. 26, 2024
(86) PCT No.: PCT/EP2024/061511

§ 371 Date: Oct. 24, 2025

(30) Foreign Application Priority Data
Apr. 26, 2023 (EP) 23170033.7

(51) Int. Cl. A61F 2/14

(52) U.S. Cl. CPC A61F 2/1451; A61F 2/148; A61F 2210/0085; A61F 2240/004

(57) ABSTRACT

The invention relates to a crosslinkable liquid composition comprising: (i) a polyethylene glycol diacrylate (PEGDA), (ii) a gelatin or gelatin-based crosslinkable biomaterial, and (iii) a photoinitiator and optionally a co-initiator, for use in a method of treating an eye disorder in an eye of a subject, wherein the method comprises applying a crosslinkable liquid composition onto an anterior corneal surface of said eye comprising said eye disorder using a mold, crosslinking the crosslinkable liquid composition on the anterior corneal surface of the eye, thereby obtaining a crosslinked composition on the anterior corneal surface of the eye, and correcting the curvature of the crosslinked composition.

### FIELD OF THE INVENTION

The invention is broadly in the field of medicine, more precisely in the field of ophthalmology. In particular, the invention concerns the use of a crosslinkable liquid composition in a method of treatment of an eye disorder, such as a refractive error or a chronic or subacute corneal disease involving an irregularity of the cornea in a subject.

### BACKGROUND OF THE INVENTION

The cornea is the transparent tissue in the front of the eye. It functions as the window of the eye and is responsible for two thirds of the refraction of the incoming light, while the remaining refraction is done by the crystalline lens. Proper refraction of incoming light is necessary to project a clear image onto the retina, where light is converted to electrical signals that are transmitted via the optic nerve to the visual cortex in the brain. Refractive disorders, such as myopia, hyperopia, astigmatism and presbyopia, can blur vision for various distances and are, in most cases, the result of an aberrant corneal curvature. A lens can be placed in front of the eye as a correction, and that can either be in the shape of contact lenses or spectacles, to compensate the aberrant light path. Uncorrected refractive errors can lead to (severe) visual impairment and, secondarily, to headaches, fatigue and eye irritation. That is why refractive errors are listed as the most prevalent cause of reversible blindness worldwide.

Another solution to correcting refractive disorders is to reshape the cornea by means of refractive laser surgery, termed laser ablation or photoablation, of which different techniques exist including photorefractive keratectomy (PRK), laser assisted in situ keratomileusis (LASIK) and small incision lenticule extraction (SMILE). The common denominator of these procedures is that they aim to adjust the curvature of the cornea by means of ablation, i.e. removing corneal tissue by means of a laser pulse. This tissue dissection will result in change of the corneal curvature and thus correcting the corneal focusing power to properly focus light onto the retina. In the case of hyperopia, the central optical zone is relatively steepened by applying laser pulses to the corneal midperiphery, while in myopia the central cornea is flattened to reduce the curvature.

Photoablative treatment for refractive errors, like PRK, LASIK and SMILE, have limitations as they are all fundamentally subtractive. Since tissue is removed from the cornea to relatively steepen or flatten the curvature, the most obvious limitation is the extent of the tissue removal required to achieve the effect. Removing too much corneal tissue renders a thin cornea that is prone to ectasia (corneal thinning) and corneal perforation. For hyperopia, photo-ablative laser surgery is safe and effective up to +2 dioptres (dpt) in practice, but becomes less predictable in higher degrees. This is also the case for astigmatism up to 3 dpt. Secondly, the effect of refractive surgery (for hyperopia or presbyopia) tends to regress over time since epithelial cells overgrow the induced grooves in the cornea, thereby returning to a state similar to its initial aberrant curvature. Thirdly, refractive surgery has, depending on the specific technique, risks associated as a result of suboptimal procedure such as risk of corneal haze and long visual recovery periods (PRK), flap related problems (LASIK) or complications due to challenging technical methods (SMILE).

In view thereof, there remains a need in the art for further and/or improved compositions and methods for treating eye disorders such as refractive errors.

### SUMMARY OF THE INVENTION

The present inventors have found a crosslinkable liquid composition and its use in a method of treating eye disorders based on the crosslinkable liquid composition that is placed on a cornea, and crosslinked in situ using UV or visible light, thereby addressing one or more of the above-mentioned problems in the art.

The present invention is at least in part based on the inventors' innovative insight and experimental evaluation that a refractive error or a chronic or subacute corneal disease or disorder involving an irregularity of the cornea can be treated by applying a crosslinkable composition onto the anterior corneal surface of the affected eye as a liquid using a mold, subsequently crosslinking the composition in situ. It is then possible, when necessary to correct the curvature of the newly formed corneal onlay, using a technique such as photoablation, without subtracting the stroma volume from the cornea per se. The advantages of crosslinking the tissue in situ are that the crosslinking causes intermolecular crosslinking of the liquid composition and that simultaneously the UV or visible light results in crosslinking (or connecting) the composition with the cornea thereby ensuring adhesion to the cornea through chemical interaction. In this way, problems which are common with lenses such as infection are avoided. Additionally, as the crosslinkable biomaterial is liquid, it perfectly fits the patient's corneal geometry compared to prefabricated onlays.

The method of the present invention provides a long-term, but reversible, solution for treating eye disorders, such as a refractive error or a chronic or subacute corneal disease involving an irregularity of the cornea. There is very limited risk of post-procedure complications such as dry eye disease or post operative pain, as the corneal stromal tissue is not affected, neither are the corneal nerves damaged. Moreover, the present method provides a wider therapeutic window than subtractive refractive laser surgery as the biomaterial is added to the corneal surface, and the method is not dependent on the thickness of the cornea itself. For example, higher corrections are possible for hyperopia and presbyopia, irregular corneas can be treated, and higher astigmatism corrections than that with just laser can be achieved. Additionally, the present method allows for restoring/recovering a partly or completely damaged cornea resulting from chronic or acute corneal diseases.

The invention thus provides methods which involve the use of a combination of a polyethylene glycol diacrylate (PEGDA), and a gelatin or gelatin-based crosslinkable biomaterial as a crosslinkable liquid composition for use in the treatment of eye disorders, whereby the cross-linkable liquid is crosslinked after application onto the surface of the eye.

Accordingly, a first aspect of the invention relates to a crosslinkable liquid composition for use in a method of treating an eye disorder in an eye of a subject, wherein the method comprises:
applying a crosslinkable liquid composition onto an anterior corneal surface of said eye comprising said eye disorder,
crosslinking the crosslinkable liquid composition on the anterior corneal surface of the eye, thereby obtaining a crosslinked composition on the anterior corneal surface of the eye, and optionally,
correcting the curvature of the crosslinked composition,
   wherein the method comprises introducing said crosslinkable liquid composition into a mold which is positioned onto the anterior corneal surface of the eye,
   wherein the eye disorder is a refractive error or a chronic or subacute corneal disease involving an irregularity of the cornea, and
   wherein the crosslinkable liquid composition comprises:
a polyethylene glycol diacrylate (PEGDA), and
a gelatin or a gelatin-based crosslinkable biomaterial.

Preferably, the invention provides a crosslinkable liquid composition for use in a method of treating an eye disorder in an eye of a subject, wherein the method comprises:
applying a crosslinkable liquid composition onto an anterior corneal surface of said eye comprising said eye disorder,
crosslinking the crosslinkable liquid composition on the anterior corneal surface of the eye, thereby obtaining a crosslinked composition on the anterior corneal surface of the eye, and
correcting the curvature of the crosslinked composition,
   wherein the method comprises introducing said crosslinkable liquid composition into a mold which is positioned onto the anterior corneal surface of the eye,
   wherein the eye disorder is a refractive error selected from the group consisting of myopia, hyperopia, astigmatism and presbyopia, and
   wherein the crosslinkable liquid composition comprises:
a polyethylene glycol diacrylate (PEGDA),
a gelatin or a gelatin-based crosslinkable biomaterial, and
a photoinitiator and optionally a co-initiator.

Optionally, the anterior corneal surface of the eye is debrided prior to applying the mold and the crosslinkable liquid composition thereon. The crosslinkable liquid composition as taught herein advantageously allows to control in the treatment method the degree of swelling of the crosslinked composition, i.e., the corneal onlay, and to protect the corneal onlay to acute and chronic degradation. Indeed, once the hydrogel forms a network and attaches to the cornea, the hydrogel will take up water (applied during the procedure as irrigation fluid, of from the underlying cornea or tear fluid) until it reaches an equilibrium. The swelling of a hydrogel will lead to significant increase in thickness of the corneal onlay. This step may be followed by ablation of the corneal onlay according to the desired optical power of the cornea, thereby correcting vision. It is critical that the onlay has a satisfactory swelling behaviour. The hydrogel should reach its equilibrium (i.e., the ultimate thickness) before the next steps are performed. After this procedure, the corneal epithelium must grow over the newly formed and ablated hydrogel, similar to the recovery period after routine surgical abrasion of the cornea. The period that the corneal onlay is exposed to the tear film of the patient, it is sensitive to enzymatic degradation since tears possess matrix metalloproteases (MMPs), enzymes that can breakdown extracellular matrix components such as gelatin or collagen. Evidence for this is seen in patients with persistent, non-healing epithelial defects where prolonged exposure to tear film enzymes and inflammation can cause degradation and loss of the underlying corneal tissue. As illustrated in the example section, the crosslinkable composition as used in the present uses or method can solve the aforementioned problems that may arise with corneal onlays with regard to the swelling and degradation behaviour.

In embodiments of the uses or methods as taught herein, the refractive error is selected from the group consisting of myopia, hyperopia, astigmatism and presbyopia.

In embodiments of the uses or methods as taught herein, the chronic or subacute corneal disease involving an irregularity of the cornea is selected from the group consisting of a corneal ulcer, a corneal erosion, a corneal ectasia, or a corneal irregularity caused by trauma or epithelial basement membrane dystrophy. Preferably, the corneal ectasia is keratoconus.

In embodiments of the uses or methods as taught herein, the gelatin-based crosslinkable biomaterial is selected from the group consisting of a gelatin methacrylate, a gelatin desaminotyrosine, a gelatin desaminotyrosyl tyrosine, a gelatin tyramine, and a thiolated gelatin.

In embodiments of the uses or methods as taught herein, the crosslinkable liquid composition comprises from about 0.5% to about 15.0% (w/v) of the PEGDA.

In embodiments of the uses or methods as taught herein, the crosslinkable liquid composition comprises from about 5.0% to about 40.0% (w/v) of the gelatin or gelatin-based crosslinkable biomaterial.

In embodiments of the uses or methods as taught herein, the PEGDA has a molecular mass of about 250 g/mol to about 6000 g/mol. Preferably, the PEGDA has a molecular mass of about 500 g/mol to about 1000 g/mol.

In embodiments of the uses or methods as taught herein, the gelatin-based crosslinkable biomaterial is a functionalized gelatin having a degree of substitution of from about 40% to about 90%.

In embodiments of the uses or methods as taught herein, the mold is a corneal vacuum suction device, a corneal bath or a contact lens.

In embodiments of the uses or methods as taught herein, the method comprises applying said mold onto the anterior corneal surface of the eye either before or after applying said crosslinkable liquid composition into said mold.

In embodiments of the uses or methods as taught herein, the method comprises removing the mold after crosslinking the crosslinkable composition.

In embodiments of the uses or methods as taught herein, the crosslinkable liquid composition is applied as a single layer.

In embodiments of the uses or methods as taught herein, the crosslinking is performed by photocrosslinking, by exposure to O₂ or by one or more enzymes; preferably wherein the crosslinking is performed by photocrosslinking such as by UV irradiation or irradiation with visible light.

In embodiments of the uses or methods as taught herein, the crosslinkable liquid composition further comprises a photoinitiator and optionally a co-initiator.

In embodiments of the uses or methods as taught herein, the crosslinkable liquid composition comprises a gelatin methacrylate, a polyethylene glycol diacrylate, and a photoinitiator; such as wherein the photoinitiator is Irgacure 2959 or Lithium phenyl-2,4,6-trimethylbenzoylphosphinate.

In embodiments of the uses or methods as taught herein, the crosslinkable liquid composition comprises a gelatin desaminotyrosine, a polyethylene glycol diacrylate, a photoinitiator, and optionally a co-initiator; such as wherein the photoinitiator comprises tris(2,2′-bipyridine) ruthenium (II) or riboflavin and the co-initiator is sodium persulphate.

In embodiments of the uses or methods as taught herein, the crosslinked composition has a diameter of from about 6.0 mm to about 9.0 mm and a thickness of from about 10.0 μm to about 400.0 μm prior to correcting the curvature of the crosslinked composition.

In embodiments of the uses or methods as taught herein, the crosslinked composition is resistant to biodegradation for a period of at least 6 months, preferably at least 12 months.

A related aspect of the invention provides a method of treating an eye disorder in an eye of a subject, wherein the method comprises:
applying a crosslinkable liquid composition onto an anterior corneal surface of said eye comprising said eye disorder,
crosslinking the crosslinkable liquid composition on the anterior corneal surface of the eye,
thereby obtaining a crosslinked composition on the anterior corneal surface of the eye; and optionally,
correcting the curvature of the crosslinked composition,
   wherein the method comprises introducing said crosslinkable liquid composition into a mold which is positioned onto the anterior corneal surface of the eye,
   wherein the eye disorder is a refractive error or a chronic or subacute corneal disease involving an irregularity of the cornea, and
   wherein the crosslinkable liquid composition comprises:
a polyethylene glycol diacrylate (PEGDA), and
a gelatin or a gelatin-based crosslinkable biomaterial.

Preferably, a related aspect of the invention provides a method of treating an eye disorder in an eye of a subject, wherein the method comprises:
applying a crosslinkable liquid composition onto an anterior corneal surface of said eye comprising said eye disorder,
crosslinking the crosslinkable liquid composition on the anterior corneal surface of the eye, thereby obtaining a crosslinked composition on the anterior corneal surface of the eye; and
correcting the curvature of the crosslinked composition,
   wherein the method comprises introducing said crosslinkable liquid composition into a mold which is positioned onto the anterior corneal surface of the eye,
   wherein the eye disorder is a refractive error selected from the group consisting of myopia, hyperopia, astigmatism and presbyopia, and
   wherein the crosslinkable liquid composition comprises:
a polyethylene glycol diacrylate (PEGDA),
a gelatin or a gelatin-based crosslinkable biomaterial, and
a photoinitiator and optionally a co-initiator.

The above and further aspects and preferred embodiments of the invention are described in the following sections and in the appended claims. The subject-matter of appended claims is hereby specifically incorporated in this specification.

### DESCRIPTION OF THE DRAWINGS

FIG. 1: Schematic representation illustrating the different steps of an exemplary method according to an embodiment of the invention for the treatment of a refractive error such as a hypermetropic cornea (farsightedness) using a mold.

FIG. 2: Schematic representation illustrating the different steps of an exemplary method according to an embodiment of the invention for the treatment of a refractive error such as a hypermetropic cornea (farsightedness) using an o-ring.

FIG. 3: Schematic representation to compare methods of refractive correction according to traditional methods and according to an embodiment of the invention.

FIG. 4: Graph illustrating the swelling of comparative gelatin hydrogels in function of molecular weight and gelatin concentration. Black full line: 160 kDa GelMA 20%-0% PEGDA, grey full line: 160 kDa GelMA 10%-0% PEGDA, grey dotted line: 90 kDa GelMA 20%-0% PEGDA, black dotted line: 90 kDa GelMA 10%-0% PEGDA; X-axis: time (hours); Y-axis: degree of swelling (%).

FIG. 5: Graph illustrating the swelling rate of a comparative gelatin hydrogel (160 kDa GelMA 20%) and crosslinked compositions according to an embodiment of the invention (160 kDa GelMA 20% supplemented with 1% and 2% of PEGDA). Grey dotted line: 160 kDa GelMA 20%-0% PEGDA, black dotted line: 160 kDa GelMA 20%-1% PEGDA, black full line: 160 kDa GelMA 20%-2% PEGDA; X-axis: time (hours); Y-axis: degree of swelling (%).

FIG. 6: Graph illustrating the swelling rate of a comparative gelatin hydrogel (160 kDa GelMA 10%) and crosslinked compositions according to an embodiment of the invention (160 kDa GelMA 10% supplemented with 1% and 2% of PEGDA). Grey dotted line: 160 kDa GelMA 10%-0% PEGDA, black dotted line: 160 kDa GelMA 10%-1% PEGDA, black full line: 160 kDa GelMA 10%-2% PEGDA; X-axis: time (hours); Y-axis: degree of swelling (%).

FIG. 7: Graph illustrating the degradation of comparative gelatin hydrogels (90 kDa GelMA 20% or 10%), a comparative PEGDA hydrogel (PEGDA 10%), and crosslinked compositions according to an embodiment of the invention (90 kDa GelMA 20% or 10% supplemented with 1% and 2% of PEGDA). Grey full line: 90 kDa GelMA 20%-0% PEGDA, grey dotted line: 90 kDa GelMA 20%-1% PEGDA, grey striped line: 90 kDa GelMA 20%-2% PEGDA, black full line: 90 kDa GelMA 10%-0% PEGDA, black dotted line: 90 kDa GelMA 10%-1% PEGDA, black striped line: 90 kDa GelMA 10%-2% PEGDA, black striped and dotted line: 10% PEGDA; X-axis: time (hours); Y-axis: residual weight (%).

FIG. 8: Graph illustrating the degradation of comparative gelatin hydrogels (160 kDa GelMA 20% or 10%), a comparative PEGDA hydrogel (PEGDA 10%), and crosslinked compositions according to an embodiment of the invention (160 kDa GelMA 20% or 10% supplemented with 1% and 2% of PEGDA). Black full line: 160 kDa GelMA 20%-0% PEGDA, black dotted line: 160 kDa GelMA 20%-1% PEGDA, black striped line: 160 kDa GelMA 20%-2% PEGDA, grey full line: 160 kDa GelMA 10%-0% PEGDA, grey dotted line: 160 kDa GelMA 10%-1% PEGDA, grey striped line: 160 kDa GelMA 10%-2% PEGDA, black striped and dotted line: 10% PEGDA; X-axis: time (hours); Y-axis: residual weight (%).

FIG. 9: Photographs illustrating (A) the crosslinkable liquid composition (1 μl) before crosslinking (FIG. 9A) and (B) the crosslinked composition after crosslinking of the crosslinkable liquid composition (FIG. 9B) on the eye of a rat.

FIG. 10: Optical coherence tomography (OCT) image illustrating an in vivo cross section of the crosslinked corneal onlay on the cornea of the eye of a rat.

FIG. 11: Photographs illustrating (A) the crosslinkable liquid composition (10 μl) before crosslinking (FIG. 11A) and (B) the crosslinked composition after crosslinking of the crosslinkable liquid composition (FIG. 11B) applied on a rabbit's eye.

FIG. 12: Optical coherence tomography (OCT) image illustrating an in vivo cross section of the crosslinked corneal onlay on the cornea of the eye of a rabbit. The soft contact lens is also visible.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed. Whereas the term “one or more”, such as one or more members of a group of members, is clear per se, by means of further exemplification, the term encompasses inter alia a reference to any one of said members, or to any two or more of said members, such as, e.g., any ≥3, >4, >5, ≥6 or ≥7 etc. of said members, and up to all said members.

All documents cited in the present specification are hereby incorporated by reference in their entirety.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

The present invention is at least in part based on the inventors' innovative insight and experimental evaluation that a refractive error or a chronic or subacute corneal disease involving an irregularity of the cornea can be treated by applying a crosslinkable composition onto the anterior corneal surface of the affected eye as a liquid using a mold for holding the crosslinkable composition while applying it to the anterior corneal surface, subsequently crosslinking the composition in situ and, optionally, correcting the curvature of the newly formed corneal onlay, such as by photoablation or Small-incision lenticule extraction (SMILE), without subtracting the stroma of the cornea per se. Accordingly, the method of present invention is less invasive as subtractive refractive laser surgery performed directly on the cornea.

The method of present invention provides an integrated, long-term, but reversible, solution for treating eye disorders, such as a refractive error or a chronic or subacute corneal disease involving an irregularity of the cornea, and has several advantageous over known methods for treating such eye disorders.

For example, unlike glasses or contact lenses, the method of present invention does not obstruct the subject during heavy duty work or contact sport, the crosslinked composition on the cornea obtained by the method present invention does not feel uncomfortable for the subject and, once applied, provides a long-term treatment of the eye disorder without risk of dry eyes and severe bacterial eye infections due to poor lens hygiene.

Furthermore, compared to subtractive refractive laser surgery performed directly on the eye, the method of the present invention has a very limited risk to post-procedure complications such as dry eye disease, post-operative pain or flap related problems, as the corneal stromal tissue is not affected nor is an epithelial flap is created. Moreover, the method of present invention provides a wider therapeutic window than subtractive refractive laser surgery performed on the cornea as biomaterial is added to the corneal surface and the method is not dependent on the thickness of the cornea itself. For example, higher corrections are possible for, for example, hyperopia and presbyopia, irregular corneas can be treated and high astigmatism correction can be achieved.

The method of present invention particularly aims at treating a subject's eye disorder which was already present prior to treating the subject with the method as taught herein and not to treat a refractive error caused by the presence of the crosslinked material applied by the method as taught herein.

By experimental testing, the present inventors have found that the crosslinkable liquid composition comprising polyethylene glycol diacrylate (PEGDA) in addition to a gelatin or gelatin-based crosslinkable biomaterial advantageously allows to obtain corneal onlays with particularly advantageous properties such as good swelling behaviour and protection against acute or chronic degradation.

The invention thus provides methods and compositions for use therein which involve the use of a combination of a polyethylene glycol diacrylate (PEGDA) and a gelatin (or gelatin-based crosslinkable biomaterial) as a crosslinkable liquid composition for use in the treatment of eye disorders, whereby the cross-linkable liquid is crosslinked after application onto the surface of the eye.

Accordingly, the invention relates to a crosslinkable liquid composition for use in a method of treating an eye disorder in an eye of a subject, wherein the method comprises:
applying a crosslinkable liquid composition onto an anterior corneal surface of said eye comprising said eye disorder, and
crosslinking the crosslinkable liquid composition on the anterior corneal surface of the eye, thereby obtaining a crosslinked composition on the anterior corneal surface of the eye.

In particular embodiments, the method may further involve correcting the curvature of the crosslinked composition.

The methods of the invention in which the composition is used typically comprise introducing said crosslinkable liquid composition into a mold which is positioned onto the anterior corneal surface of the eye and thereafter crosslinking the liquid composition.

In preferred embodiments of the invention, the eye disorder is a refractive error or a chronic or subacute corneal disease involving an irregularity of the cornea.

The methods of the invention are characterized in that the crosslinkable liquid composition comprises:
a polyethylene glycol diacrylate (PEGDA), and
a gelatin or gelatin-based crosslinkable biomaterial, and
a photoinitiator and optionally a co-initiator.

A related aspect of the invention provides a method of treating an eye disorder, such as a refractive error or a chronic or subacute corneal disease involving an irregularity of the cornea, in an eye of a subject, wherein the method comprises the steps of:
applying a crosslinkable liquid composition comprising a polyethylene glycol diacrylate (PEGDA) and a gelatin or gelatin-based crosslinkable biomaterial onto an anterior corneal surface of said eye comprising said eye disorder, and
crosslinking the crosslinkable liquid composition on the anterior corneal surface of the eye, thereby obtaining a crosslinked composition on the anterior corneal surface of the eye, thereby treating said eye disorder.

Optionally, the method can comprise the step of correcting the curvature of the crosslinked composition, after said step of crosslinking the crosslinkable liquid.

Hence, in embodiments, the method comprises the steps of:
applying a crosslinkable liquid composition comprising a polyethylene glycol diacrylate (PEGDA) and a gelatin or gelatin-based crosslinkable biomaterial onto an anterior corneal surface of said eye comprising said eye disorder,
crosslinking the crosslinkable liquid composition on the anterior corneal surface of the eye, thereby obtaining a crosslinked composition on the anterior corneal surface of the eye, and
correcting the curvature of the crosslinked composition, thereby treating said eye disorder.

The method typically comprises introducing said crosslinkable liquid composition into a mold which is positioned onto the anterior corneal surface of the eye.

Related aspects provide:
the use of a crosslinkable liquid composition for the manufacture of a medicament for the treatment of an eye disorder in an eye of a subject, wherein the method comprises: applying a crosslinkable liquid composition onto an anterior corneal surface of said eye comprising said eye disorder, crosslinking the crosslinkable liquid composition on the anterior corneal surface of the eye, thereby obtaining a crosslinked composition on the anterior corneal surface of the eye, and optionally, correcting the curvature of the crosslinked composition, wherein the method comprises introducing said crosslinkable liquid composition into a mold which is positioned onto the anterior corneal surface of the eye, wherein the eye disorder is a refractive error or a chronic or subacute corneal disease involving an irregularity of the cornea, and wherein the crosslinkable liquid composition comprises: (i) a polyethylene glycol diacrylate (PEGDA), and (ii) a gelatin or gelatin-based crosslinkable biomaterial.
the use of a crosslinkable liquid composition for the treatment of the treatment of an eye disorder in an eye of a subject, wherein the method comprises: applying a crosslinkable liquid composition onto an anterior corneal surface of said eye comprising said eye disorder, crosslinking the crosslinkable liquid composition on the anterior corneal surface of the eye, thereby obtaining a crosslinked composition on the anterior corneal surface of the eye, and optionally, correcting the curvature of the crosslinked composition, wherein the method comprises introducing said crosslinkable liquid composition into a mold which is positioned onto the anterior corneal surface of the eye, wherein the eye disorder is a refractive error or a chronic or subacute corneal disease involving an irregularity of the cornea, and wherein the crosslinkable liquid composition comprises: (i) a polyethylene glycol diacrylate (PEGDA), and (ii) a gelatin or gelatin-based crosslinkable biomaterial.

Preferably, related aspects provide:
the use of a crosslinkable liquid composition for the manufacture of a medicament for the treatment of an eye disorder in an eye of a subject, wherein the method comprises: applying a crosslinkable liquid composition onto an anterior corneal surface of said eye comprising said eye disorder, crosslinking the crosslinkable liquid composition on the anterior corneal surface of the eye, thereby obtaining a crosslinked composition on the anterior corneal surface of the eye, and correcting the curvature of the crosslinked composition, wherein the method comprises introducing said crosslinkable liquid composition into a mold which is positioned onto the anterior corneal surface of the eye, wherein the eye disorder is a refractive error selected from the group consisting of myopia, hyperopia, astigmatism and presbyopia, and wherein the crosslinkable liquid composition comprises: (i) a polyethylene glycol diacrylate (PEGDA), (ii) a gelatin or gelatin-based crosslinkable biomaterial, and (iii) a photoinitiator and optionally a co-initiator.
the use of a crosslinkable liquid composition for the treatment of an eye disorder in an eye of a subject, wherein the method comprises: applying a crosslinkable liquid composition onto an anterior corneal surface of said eye comprising said eye disorder, crosslinking the crosslinkable liquid composition on the anterior corneal surface of the eye, thereby obtaining a crosslinked composition on the anterior corneal surface of the eye, and correcting the curvature of the crosslinked composition, wherein the method comprises introducing said crosslinkable liquid composition into a mold which is positioned onto the anterior corneal surface of the eye, wherein the eye disorder is a refractive error selected from the group consisting of myopia, hyperopia, astigmatism and presbyopia, and wherein the crosslinkable liquid composition comprises: (i) a polyethylene glycol diacrylate (PEGDA), (ii) a gelatin or gelatin-based crosslinkable biomaterial, and (iii) a photoinitiator and optionally a co-initiator.

Reference to “therapy” or “treatment” encompasses curative treatments, and the terms may particularly refer to the alleviation or measurable lessening of one or more symptoms or measurable markers of a pathological condition such as a disease or disorder or a dysfunction (e.g. as a result of trauma or surgery). Measurable lessening includes any statistically significant decline in a measurable marker or symptom. Generally, the terms encompass both curative treatments and treatments directed to reduce symptoms and/or slow progression of the disease.

The term eye disorder thus generally encompasses both conditions caused by disease and conditions caused by other factors such as trauma or operation, which can affect the normal functioning of the eye.

The terms “subject”, “individual” or “patient” are used interchangeably throughout this specification, and typically and preferably denote humans, but may also encompass reference to non-human animals, preferably warm-blooded animals, even more preferably non-human mammals. Particularly preferred are human subjects including both genders and all age categories thereof. In other embodiments, the subject is an experimental animal or animal substitute as a disease model. The term does not denote a particular age or sex.

The term “subject in need of treatment” or similar as used herein refers to subjects diagnosed with or having a disease or disorder as recited herein.

In embodiments, the eye disorder is a refractive error. In embodiments of the uses or methods as taught herein, the refractive error is selected from the group consisting of myopia, hyperopia, astigmatism (e.g. regular or irregular astigmatism, corneal surgery-induced astigmatism or corneal transplantation-induced astigmatism), and presbyopia.

The term “refractive error” or “refraction error” as used herein refers to an eye disorder wherein the shape of the eye and/or cornea prevents light from focussing correctly on the retinal fovea leading to impaired vision. Non-limiting examples of refractive errors are nearsightedness (myopia), farsightedness (hyperopia), regular and irregular astigmatism, corneal surgery-induced astigmatism, corneal transplantation-induced astigmatism, and presbyopia. Symptoms caused by refractive errors include, but are not limited to, double vision, hazy vision, seeing a glare or halo around bright lights, squinting, headaches and eye strains. A refractive error may be diagnosed by any means known in the art, such as by use of an automated refractor. In preferred embodiments, the refractive error is of primary cause (e.g. naturally occurring or non-surgically induced) or of secondary cause which may include, but not limited to surgically induced (e.g. post-LASIK ectasia or post corneal transplantation such as full thickness penetrating keratoplasty) as a result from infection, genetic factors, degenerative eye diseases or trauma to the eye.

The term “myopia” or “nearsightedness” as used herein has its meaning as generally accepted in the art. In myopia or nearsightedness, light rays are brought to focus in front of the retina. This may occur because the focusing power of the cornea and lens is very high and/or because the eyeball is too long from front to back. For myopia, subtractive photo-ablative laser surgery is safe and effective up to −10 diopters (dpt) in practice, but becomes less predictable in higher degrees of myopia and display higher complication rates. Accordingly, in particular embodiments, the refractive error is myopia with a diopter of more than −10.0, such as from −10.5 to −20.0.

The term “hyperopia” or “farsightedness” as used herein has its meaning as generally accepted in the art. In hyperopia or farsightedness, light rays are brought to focus behind the retina. This may occur because the focusing power of the cornea and lens is very low and/or because the eyeball is short in length from front to back. For hyperopia, subtractive photo-ablative laser surgery is safe and effective up to 3 diopters (dpt) in practice, but becomes less predictable in higher diopters. Accordingly, in particular embodiments, the refractive error is hyperopia with a diopter of more than +3.0, such as from +3.5 to +10.0, from +3.5 to +8.0, preferably from +3.5 to +4.0, +3.5, or +4.0.

The term “presbyopia” or “age-related farsightedness” as used herein has its meaning as generally accepted in the art. Presbyopia is physiological insufficiency of accommodation associated with the aging of the eye that results in progressively worsening ability to focus clearly on close objects.

Presbyopia typically occurs due to age related changes in lens (decreased elasticity and increased hardness) and ciliary muscle of the eye, causing the eye to focus light behind rather than on the retina, when looking at close objects.

The term “astigmatism” as used herein has its meaning as generally accepted in the art, and includes regular, irregular astigmatism, simple astigmatism, compound astigmatism, myopic astigmatism, hyperopic astigmatism, mixed astigmatism, lenticular astigmatism, and corneal astigmatism. Astigmatism is a refractive error in the eye due to rotational asymmetry in the eye's refractive power. The underlying mechanism involves an irregular curvature of the cornea or abnormalities in the lens of the eye. In eyes without astigmatism, the cornea and lens have a more or less similar curvature in all directions. This allows light to be focused to a single point on the retina. People with astigmatism have more curvature in one direction, or meridian, than in another, so that light is not able to focus on a single point on the retina. This results in blurring of vision at all distances. For astigmatism, subtractive photo-ablative laser surgery is safe and effective up to 3 diopters (dpt) in practice, but becomes less predictable in higher diopters. Accordingly, in particular embodiments, the refractive error is astigmatism with a diopter of more than +3.0, such as from +3.5 to +16.0, from +3.5 to +8.0, preferably from +3.5 to +5.0, like +3.5, +4.0, +4.5, or +5.0. There are different forms of corneal astigmatism. The corneal astigmatism may be corneal surgery-induced astigmatism or corneal transplantation-induced astigmatism.

Preferably, the astigmatism such as corneal astigmatism is not a surgery-induced or surgery-related astigmatism. In embodiments, the astigmatism such as corneal astigmatism is not a surgical astigmatism caused by sutures, e.g., leading to refractive complications. In embodiments, the astigmatism such as corneal astigmatism has a congenital origin.

In embodiments, the astigmatism may not be caused by an invasive treatment for correcting an eye disorder. Accordingly, in embodiments, the astigmatism is not caused by refractive laser surgery, such as PRK, LASIK or SMILE.

In embodiments, the eye disorder is a chronic or subacute corneal disease involving an irregularity of the cornea. Non-limiting examples of chronic or subacute corneal disease involving an irregularity of the cornea comprise corneal ulcers (e.g. caused by trauma or inflammation), corneal erosion, corneal ectactic disorders (i.e. corneal thinning) such as keratoconus, keratoglobus or post-LASIK ectasia, or a corneal irregularity inducing an irregular astigmatism such as a result from trauma or epithelial basement membrane dystrophy.

In embodiments of the uses or methods as taught herein, the chronic or subacute corneal disease involving an irregularity of the cornea is selected from the group consisting of a corneal ulcer, a corneal erosion, a corneal ectasia, or a corneal irregularity caused by trauma or epithelial basement membrane dystrophy. Preferably, the corneal ectasia is keratoconus.

The terms “corneal ectasia” or “corneal ectatic disorder” as used herein refers to a group of uncommon, noninflammatory eye disorders characterised by bilateral thinning of the central, paracentral, or peripheral cornea.

In embodiments, the corneal ectasia may be selected from the group consisting of keratoconus, keratoglobus, pellucid marginal degeneration, posterior keratoconus, post-LASIK ectasia, and Terrien's marginal degeneration.

The term “keratoconus” refers to a progressive, noninflammatory, bilateral, asymmetric disease, characterized by paraxial stromal thinning and weakening that leads to corneal surface distortion. In embodiments of the uses or methods as taught herein, the chronic or subacute corneal disease involving an irregularity of the cornea may be caused by an invasive treatment for correcting an eye disorder. Hence, in embodiments, the chronic or subacute corneal disease involving an irregularity of the cornea may be caused by refractive laser surgery, such as PRK, LASIK or SMILE.

Accordingly, in embodiments, the subject received an invasive treatment for correcting the eye disorder in the eye, such as a refractive error, prior to the application of the crosslinkable liquid composition. In more particular embodiments, the subject received refractive laser surgery, such as PRK, LASIK or SMILE, prior to the application of the crosslinkable liquid composition onto the anterior corneal surface of the eye.

In alternative embodiments, the chronic or subacute corneal disease involving an irregularity of the cornea may not be caused by an invasive treatment for correcting an eye disorder. Accordingly, in embodiments, the chronic or subacute corneal disease involving an irregularity of the cornea is not caused by refractive laser surgery, such as PRK, LASIK or SMILE.

In other embodiments, the subject did not receive any invasive treatment for correcting the eye disorder in the eye, such as a refractive error, prior to the application of the crosslinkable liquid composition. In more particular embodiments, the subject did not receive refractive laser surgery, such as PRK, LASIK or SMILE, prior to the application of the crosslinkable liquid composition onto the anterior corneal surface of the eye.

Of particular interest are those subjects for which the eye disorder cannot be treated using existing techniques, such as subjects that are excluded from treatment by refractive laser therapy, such as subjects with a very thin cornea, subjects with persistent dry eyes or subjects performing contact sports. Accordingly, in embodiments, the subject has a cornea thickness of less than 480.0 μm, less than 450.0 μm or less than 400.0 μm.

Crosslinking is the formation of chemical links between molecular chains to form a three-dimensional network of connected molecules. Crosslinks may be formed by chemical reactions that occur spontaneously or are initiated by, for example, one or more enzymes, heat, pressure, change in pH, or irradiation. These chemical reactions may also be initiated by the presence of one or more crosslinking agents such as photoinitiators, which typically comprises multiple functional groups and form radicals upon irradiation, thereby as such starting the crosslinking reaction.

The “crosslinkable liquid” or “crosslinkable liquid composition” as referred to herein comprises crosslinkable biocompatible material (e.g. biomaterial). The terms “liquid” or “liquid composition” in the context of the present invention encompass both completely liquid and semi-liquid compositions, i.e. include compositions which have a consistency between solid and liquid.

The crosslinkable liquid composition to be used in the method of present invention comprises:
a polyethylene glycol diacrylate (PEGDA), and
a gelatin or gelatin-based crosslinkable biomaterial.

Preferably, the crosslinkable liquid composition to be used in the method of present invention comprises:
a polyethylene glycol diacrylate (PEGDA),
a gelatin or gelatin-based crosslinkable biomaterial, and
a photoinitiator. The crosslinkable liquid composition optionally comprises a co-initiator.

The crosslinkable liquid composition to be used in the method of present invention advantageously comprises a combination of polymers that are able to crosslink (spontaneously or upon induction, such as by UV irradiation) and can take up substantial amounts of water from its surroundings without dissolving at body temperature, such at about 37° C. Accordingly, in particular embodiments, the step of crosslinking is carried out by photocrosslinking, more particularly by UV irradiation.

The terms “polyethylene glycol diacrylate”, “poly(ethylene glycol) diacrylate”, “PEG diacrylate” or “PEGDA” can be used interchangeably herein. PEGDA has C₃H₃O(OCH₂CH₂)ₙC₃H₃O₂ as linear formula and 26570-48-9 as CAS number.

In embodiments of the uses or methods as taught herein, the PEGDA has a molecular mass of about 250 g/mol to about 6000 g/mol. For instance, the PEGDA has a molecular mass of about 300 g/mol to about 5000 g/mol, about 350 g/mol to about 4000 g/mol, about 400 g/mol to about 3000 g/mol, about 450 g/mol to about 2000 g/mol, or about 500 g/mol to about 1000 g/mol. In embodiments, the PEGDA has a molecular mass of about 3000 g/mol to about 5000 g/mol or about 3500 g/mol to about 4500 g/mol Preferably, the PEGDA has a molecular mass of about 500 g/mol to about 1000 g/mol, such as about 600 g/mol to about 900 g/mol, or about 700 g/mol to about 800 g/mol. When reference is made to the molecular mass of PEGDA, this refers to the molecular mass of the PEG component of PEGDA. Such molecular masses of PEGDA allow satisfactory swelling and good resistance to enzymatic degradation.

Suitable poly(ethylene glycol) diacrylate includes for instance poly(ethylene glycol) diacrylate commercially available from Merck KGaA, Darmstadt, Germany under product number 455008 (average Mn 700), 437441 (average Mn 575), or 475629 (average Mn 250). Suitable polyethylene glycol diacrylate includes for instance polyethylene glycol diacrylate commercially available from Polysciences Inc., PA, USA under product number 01871 (average Mn 400 or PEGDA 400) or 25485 (average Mn 1000 or PEGDA 1000). Other suitable PEGDA (more that 80% acrylated) is available from Advanced BioMatrix, Inc. with molecular weights 1000, 3400, 6000, 10000 and 20000. The term “gelatin” refers to a composition comprising or consisting of proteins and obtained by partial hydrolysis of collagen extracted from the skin, bones, and connective tissues of animals.

The “gelatin-based crosslinkable biomaterial” refers to a crosslinkable biomaterial based on gelatin. Gelatin-based biomaterials may be GelCORE such as described in Ehsan Shirzaei Sani et al., Sutureless repair of corneal injuries using naturally derived bioadhesive hydrogels, Science Advances, 2019, Vol. 5, no. 3, or methacrylated thermoresponsive COMatrix such as described in Ghasem Yazdanpanah et al., A light-curable and tunable extracellular matrix hydrogel for in situ suture-free corneal repair, Advanced Functional Materials, 2022.

The gelatin-based crosslinkable biomaterial may be a functionalized gelatin.

In embodiments of the uses or methods as taught herein, the gelatin-based crosslinkable biomaterial may be a functionalized gelatin having a degree of substitution of from about 40% to about 90%. For instance, the gelatin-based crosslinkable biomaterial may be a functionalized gelatin having a degree of substitution of from about 50% to about 80%, or from about 60% to about 70%.

The degree of substitution of a functionalized gelatin refers to the ratio (expressed as a percentage) of the number of modified amino groups to the number of free amino groups of the gelatin.

In embodiments, the gelatin-derived crosslinkable biomaterial may be functionalized using methacrylic acid, diacrylate, diacrylamide, desaminotyrosine, desaminotyrosyl tyrosine, tyramine, or thiol (—SH).

The terms “methacrylic acid”, “2-methyl-2-propenoic acid”, “a-methacrylic acid”, “2-methylacrylic acid”, “2-methylpropenoic acid” can be used interchangeably herein. Methacrylic acid has C₄H₆O₂ as chemical formula, 2-methylprop-2-enoic acid as preferred IUPAC name, and 79-41-4 as CAS number.

The terms “desaminotyrosine”, “DAT”, “phloretic acid”, “phloretate” or “hydro-p-coumaric acid” can be used interchangeably herein. Desaminotyrosine has 3-(4-hydroxyphenyl) propanoic acid as IUPAC name.

The terms “desaminotyrosyl tyrosine”, “desaminotyrosyl-tyrosine” or “DATT” may be used interchangeably. The IUPAC name is (2R,4S)-4-amino-5-(4-hydroxyphenyl)-2-[(4-hydroxyphenyl)methyl]-3-oxopentanoic acid.

The terms “tyramine” or “4-hydroxyphenethylamine” can be used interchangeably herein.

Tyramine has 4-(2-aminoethyl) phenol as the IUPAC name.

In embodiments of the uses or methods as taught herein, the gelatin-based crosslinkable biomaterial may be selected from the group consisting of a gelatin methacrylate, a gelatin desaminotyrosine, a gelatin desaminotyrosyl tyrosine, a gelatin tyramine, and a thiolated gelatin.

The terms “gelatin methacrylate”, “gelatin methacryloyl”, “gelatin methacrylamide”, “GelMA” or “GelMa” may be used interchangeably herein.

Suitable gelatin methacrylate includes for instance gelatin methacrylate commercially available from Merck KGaA, Darmstadt, Germany under product number 900629 (gel strength 300 g Bloom, 40% degree of substitution), 900622 (gel strength 300 g Bloom, 60% degree of substitution), 900628 (gel strength 90-110 g Bloom, 60% degree of substitution) or 900496 (gel strength 300 g Bloom, 80% degree of substitution).

The terms “gelatin desaminotyrosine”, “gelatin functionalized with desaminotyrosine” or “GelDAT” may be used interchangeably herein.

Suitable gelatin desaminotyrosine includes X-Pure GelDAT commercially available from Rousselot BV, Ghent, Belgium.

The term “gelatin desaminotyrosyl tyrosine”, “gelatin functionalized with desaminotyrosyl tyrosine” or “GelDATT” may be used interchangeably herein.

Suitable gelatin desaminotyrosyl tyrosine can be synthesized as described in Roch et al. (2011, Macromol. Symp., 309, 182-189), in particular on p. 184, Materials and Methods, Functionalization of gelatin. For instance, desaminotyrosine or desaminotyrosyl tyrosine (29 mmol) may be activated by reaction with 1-ethyl-3-(3-dimethyl-aminopropyl) carbodiimide (EDC) (32 mmol) and N-hydroxysuccinimide (NHS) (43 mmol) in 110 ml of dimethyl sulfoxide (DMSO) at 37° C. After 3 h, β-mercaptoethanol (43 mmol) may be added. A gelatin solution (15 g in 150 mL DMSO) may be added and the mixture stirred at 37° C. for 5 h. The functionalized product may be precipitated in ethanol, filtered, washed with ethanol and acetone, and dried under vacuum.

The terms “gelatin tyramine”, “gelatin functionalized with tyramine” or “GTA” may be used interchangeably herein.

Suitable gelatin tyramine can be synthesized as described in Sakai et al. (2009, Biomaterials, 30, 3371-3377), in particular on p. 3372, Materials and methods, 2.2 Modification of gelatin to incorporate phenol groups, or as described in Li et al. (2015, Acta Biomater., 13, 88-100), in particular in 2. Materials and methods, 2.2 Synthesis of gelatin/tyramine/heparin (G/T/H) conjugates. For instance, gelatin derivatives possessing phenol (Ph) groups may be synthesized by combining gelatin and tyramine hydrochloride via the carbodiimide-mediated condensation of the carboxyl groups of gelatin and the amino groups of tyramine. Gelatin powder may be suspended at 2% (w/v) in a 50 mM morpholinoethanesulfonic acid (MES) aqueous solution and heated to 60° C. After dissolution of gelatin, the solution may be cooled to 25° C. To this solution, tyramine hydrochloride, EDC and NHS may be added and the solution may be stirred at 25° C. After 12 h of stirring, 50 mM sodium phosphate may be added. After a further 30 min of stirring, the resultant polymer solution may be dialyzed against deionized water, using an ultrafiltration membrane (MWCO: 10,000), until an absorbance peak at 275 nm, attributed to the presence of residual tyramine, is undetectable in the filtered solution. The sample may subsequently be lyophilized.

The terms “thiolated gelatin”, “thiol functionalized gelatin”, “thiol gelatin”, “gelatin thiol” or “Gel-SH” may be used interchangeably herein.

Suitable thiolated gelatin includes thiol functionalized gelatin commercially available from Merck KGaA, Darmstadt, Germany under product number 904643.

In embodiments, the crosslinkable liquid composition has a transparency of at least 50%, when measured over the visual spectrum (400-750 nm), when crosslinked.

In embodiments of the uses or methods as taught herein, the crosslinkable liquid composition may comprise from about 0.5% to about 15.0% (w/v) of the PEGDA. In embodiments, the crosslinkable liquid composition may comprise from about 0.5% to about 10.0% (w/v), from about 0.5% to about 5.0% (w/v) of the PEGDA, from about 0.5% to about 4.0% (w/v), from about 0.5% to about 3.0% (w/v), or from about 0.5% to about 2.0% (w/v). In embodiments, the crosslinkable liquid composition may comprise from about 1.0% to about 15.0% (w/v), from about 1.0% to about 10.0% (w/v), from about 1.0% to about 5.0% (w/v) of the PEGDA, from about 1.0% to about 4.0% (w/v), from about 1.0% to about 3.0% (w/v), or from about 1.0% to about 2.0% (w/v). Such concentrations of PEGDA allow satisfactory swelling and good resistance to enzymatic degradation.

The terms “% w/v”, “% m/v”, “percentage weight per volume” or “percentage mass per volume” may be used interchangeably and refer to the ratio of the mass of a solid (the solute) (in grams) to the volume of the solution (in ml) times 100. For instance, the crosslinkable liquid composition may comprise about 1 g of PEGDA per 100 ml of solution. If 1 g of PEGDA is used to make up a total volume of 100 ml, then a 1% w/v solution of PEGDA has been made.

In embodiments of the uses or methods as taught herein, the crosslinkable liquid composition may comprise at least about 1.0% (w/v) of the gelatin or gelatin-based crosslinkable biomaterial. In embodiments, the crosslinkable liquid composition may comprise at least about 5.0% (w/v), at least about 10.0% (w/v), at least about 15.0% (w/v), or at least about 20.0% (w/v) of the gelatin or gelatin-based crosslinkable biomaterial. Such concentrations of the gelatin or gelatin-based crosslinkable biomaterial allow satisfactory swelling and good resistance to enzymatic degradation.

In embodiments, the crosslinkable liquid composition may comprise from about 1.0% to about 40.0% (w/v), from about 1.0% to about 30.0% (w/v), from about 1.0% to about 20.0% (w/v), from about 1.0% to about 15.0% (w/v), or from about 1.0% to about 10.0% (w/v) of the gelatin or gelatin-based crosslinkable biomaterial. In embodiments of the uses or methods as taught herein, the crosslinkable liquid composition may comprise from about 5.0% to about 40.0% (w/v) of the gelatin or gelatin-based crosslinkable biomaterial. In embodiments, the crosslinkable liquid composition may comprise from about 5.0% to about 30.0% (w/v), from about 5.0% to about 20.0% (w/v), from about 5.0% to about 15.0% (w/v), or from about 10.0% to about 20.0% (w/v) of the gelatin or gelatin-based crosslinkable biomaterial. Such concentrations of the gelatin or gelatin-based crosslinkable biomaterial allow satisfactory swelling and good resistance to enzymatic degradation.

In embodiments of the uses or methods as taught herein, the gelatin or gelatin-based crosslinkable biomaterial has a molecular mass of about 50000 g/mol (50 kDa) to about 200000 g/mol (200 kDa). For instance, the gelatin or gelatin-based crosslinkable biomaterial has a molecular mass of about 60 kDa to about 190 kDa, about 70 kDa to about 180 kDa, or about 80 kDa to about 170 kDa. Preferably, the gelatin or gelatin-based crosslinkable biomaterial has a molecular mass of about 90 kDa to about 160 kDa. In further embodiments the gelatin functionalized with one or more tyrosine-derived or phenol groups has a molecular mass of about 90 kDa and/or about 160 kDa. Such molecular masses of the gelatin or gelatin-based crosslinkable biomaterial allow satisfactory swelling and good resistance to enzymatic degradation.

In embodiments, the crosslinkable liquid composition as taught herein may further comprise an aqueous solution, in particular a buffer solution, such as phosphate buffered saline (PBS).

The term “aqueous solution” refers to any solution comprising water or in which the solvent is water. Additionally, “aqueous solution” is used to describe solutions displaying commonalities to water or watery solutions, not limited to characteristics such as appearance, smell, colour, taste, viscosity, pH, absorbance, or physical state under particular temperatures.

The term “buffer component”, “buffer solution”, or “buffer” as used interchangeably herein refers to an aqueous solution comprising a mixture of a weak acid and its conjugate base or vice versa. Buffer solutions are characterized by their means of keeping the pH of a solution nearly constant when limited amounts of strong acids or strong bases are added to the solution. The amount of strong acid or strong base that can be added to the buffer solution before a significant pH change occurs is dependent on the specific buffer solution used and is commonly referred to as the buffer capacity. The pH of a buffer solution can be estimated using the Henderson-Hasselbalch equation, which is known to a person skilled in the art.

In certain embodiments, the crosslinkable liquid composition as taught herein may comprise from about 10% to about 98% by weight of an aqueous solution, in particular a buffer solution such as PBS. In embodiments, the crosslinkable liquid composition as taught herein may comprise from about 20% to about 98%, from about 30% to about 98%, from about 40% to about 98%, from about 50% to about 98%, from about 60% to about 98%, from about 60% to about 95% or from about 60% to about 90% by weight of an aqueous solution, in particular a buffer solution such as PBS.

The terms “weight percentage”, “mass percentage”, “percentage (%) by weight”, “weight %” or “wt %” indicate the mass of a substance to the total mass of the formulation (i.e. mass fraction) with a denominator of 100. Unless indicated otherwise, the wt % is provided herein compared to the total weight of the crosslinkable liquid composition.

In embodiments, the crosslinkable liquid composition as taught herein may not comprise organic solvents. In embodiments, the crosslinkable liquid composition as taught herein may be prepared in an aqueous carrier without the use of organic solvents.

The crosslinkable liquid composition may further also comprise one or more crosslinking agents and/or one or more photoinitiators, that participate in the crosslinking reaction.

In embodiments of the uses or methods as taught herein, the crosslinking may performed by photocrosslinking, by exposure to O₂ or by one or more enzymes; preferably the crosslinking is performed by photocrosslinking such as by UV irradiation or irradiation with visible light.

In embodiments, the crosslinkable liquid composition may be capable of crosslinking by photocrosslinking such as UV irradiation, by exposure to O₂ or by one or more enzymes. In preferred embodiments, the crosslinkable liquid composition is capable of crosslinking by photocrosslinking or enzymatic crosslinking. Therefore, the crosslinkable liquid composition possesses reactive functionalities that form short oligomer/polymer chains between the macromolecule chains. In embodiments of the uses or methods as taught herein, the crosslinkable liquid composition may further comprise a photoinitiator and optionally a co-initiator. In embodiments, the crosslinkable liquid composition may further comprise a photoinitiator. In embodiments, the crosslinkable liquid composition may further comprise a photoinitiator and a co-initiator.

Photoinitiators are compounds that upon radiation of light decompose into reactive species that activate polymerization of specific functional groups on the crosslinkable biomaterial. Accordingly, photoinitiators are typically used herein when the crosslinkable liquid composition is capable of being crosslinked by photocrosslinking. The type of photoinitiator as well as the concentration thereof can be varied in the crosslinkable liquid composition as intended herein. Each specific photoinitiator is typically linked to an excitation wavelength spectrum, of which the peak of the spectrum is the most optimal wavelength to create radicals upon excitation. Non-limiting examples of photoinitiators that may be used in the uses or methods as taught herein include riboflavin, indocyanine green, Janus green, rose Bengal, methylene blue, sodium persulphate, ruthenium, 2,4,6-trimethylbenzoyl)-phosphine oxide (TPO), Irgacure 2959, Lithium phenyl-2,4,6-trimethylbenzoylphosphinate (LAP), or a combination thereof.

In embodiments, the photoinitiator may be a photoinitiator that can be excited with visible light. Non-limiting examples of photoinitiators that can be excited with visible light include 2,4,6-trimethylbenzoyl)-phosphine oxide (TPO), Lithium phenyl-2,4,6-trimethylbenzoylphosphinate (LAP), flavins such as riboflavin, rose bengal or a sulfinate or sulfonate such as sodium persulphate.

In embodiments, the photoinitiator may be a photoinitiator that can be excited in the UV spectrum, hence, at a wavelength of from 250 to 450 nm, which may also be referred to as “blue light”. Non-limiting examples of photoinitiators that can be excited with UV irradiation are Irgacure 2959, Lithium phenyl-2,4,6-trimethylbenzoylphosphinate (LAP).

In embodiments, the one or more photoinitiators are water-soluble photoinitiators. Non-limiting examples are photoinitiators comprising tris(2,2′-bipyridine) ruthenium (II) such as tris(2,2′-bipyridyl)dichlororuthenium (II) hexahydrate, or other photoinitiators such as Lithium phenyl-2,4,6-trimethylbenzoylphosphinate (LAP), 2,4,6-trimethylbenzoyl)-phosphine oxide (TPO), and other members of the Irgacure photoinitiator family.

The terms “tris(2,2′-bipyridyl)dichlororuthenium (II) hexahydrate”, “tris(2,2-bipyridyl)dichlororuthenium (II) hexahydrate”, “Ru(BPY)₃”, “ruthenium-tris(2,2′-bipyridyl)dichloride”, “tris(2,2′-bipyridyl) ruthenium (II) chloride hexahydrate” may be used interchangeably herein. The CAS number is 50525-27-4. IUPAC name is 2-pyridin-2-ylpyridine; ruthenium (2+); dichloride; hexahydrate (as computed by Lexichem TK 2.7.0, PubChem release 2021.05.07).

In embodiments, the crosslinkable composition comprises from 0.05 to 2.0% (w/v), more particularly from 0.5 to 20% such as from 1.0 to 2.0% (w/v), of one or more photoinitiators.

In embodiments, the co-initiator may be a sulfinate or sulfonate such as sodium persulphate.

In embodiments of the uses or methods as taught herein, the crosslinkable liquid composition comprises a gelatin methacrylate, a polyethylene glycol diacrylate, and a photoinitiator; such as wherein the photoinitiator is Irgacure 2959 or Lithium phenyl-2,4,6-trimethylbenzoylphosphinate (LAP).

In embodiments of the uses or methods as taught herein, the crosslinkable liquid composition comprises a gelatin desaminotyrosine, a polyethylene glycol diacrylate, a photoinitiator, and optionally a co-initiator; such as wherein the photoinitiator comprises tris(2,2′-bipyridine) ruthenium (II) or riboflavin (e.g. for crosslinking the GelDAT) and Irgacure 2595 or LAP (e.g., for crosslinking the PEGDA), and the co-initiator is sodium persulphate. In embodiments of the uses or methods as taught herein, the crosslinkable liquid composition comprises a gelatin desaminotyrosine, a polyethylene glycol diacrylate, tris(2,2′-bipyridyl)dichlororuthenium (II) hexahydrate, Irgacure 2595, and sodium persulphate.

In embodiments, the crosslinkable liquid composition may further comprise one or more therapeutic agents (e.g. an analgesic, an anti-inflammatory agent, an antibiotic, a growth factor to stimulate epithelialization, or a steroid), and/or other agents such as colorants.

Once the crosslinkable liquid composition has been crosslinked, the crosslinked composition preferably does not interfere with the normal functionality of the eye and provides sufficient nutrient and gas exchange to maintain a viable corneal epithelium and stroma. Accordingly, in embodiments, the crosslinked composition is permeable to water, nutrients, oxygen, therapeutic agents (e.g. an analgesic, an anti-inflammatory agent, an antibiotic, a growth factor to stimulate epithelialization, or a steroid), and/or growth factors (e.g. exogenous or endogenous growth factors, such as nerve growth factor (NGF)).

Preferably, the crosslinked composition is compatible with clinical imaging techniques, such as clinical corneal investigation using a refractometer, optical coherence tomography, Scheimpflug tomography, Placido based tomography device or in vivo confocal imaging.

Accordingly, in embodiments, the crosslinkable liquid composition and/or crosslinked composition has a transparency of at least 40%, at least 50%, at least 60%, at least 70%, at least 80% or at least 90%, when measured using light with a wavelength spectrum that is representative of the visual spectrum (400-750 nm), when crosslinked. The transparency may be determined by any means in the art, such as by a microplate reader or spectrophotometer such as described by Rizwan et al. (Biomaterials, 2017, 120:139-154) or by Van Hoorick et al. (Adv. Healthcare Materials, 2020, 9 (6): 2000760).

In embodiments, the crosslinkable liquid composition and/or crosslinked composition has a refractive index similar to that of the native corneal stroma. The refractive index may be measured by any means in the art, such as by use of a refractometer.

Upon crosslinking of the crosslinkable liquid composition, the crosslinked composition will hold water within its three-dimensional network of polymers, resulting in the formation of a hydrogel.

The term “hydrogel” as used herein has its meaning as known in the art and refers to a biphasic material, a mixture of porous, permeable solids and at least 10% by weight or volume of interstitial fluid composed completely or mainly by water. In hydrogels the porous permeable solid is a water insoluble three dimensional network of polymers and a fluid, having absorbed a large amount of water or biological fluids. The term “hydrogel” may be used interchangeably herein with the term “crosslinked composition”.

The “swelling ratio” may be defined as the fractional increase in the weight of the crosslinked composition due to water absorption. The swelling ratio may be influenced by the type of the gelatin or gelatin-based crosslinkable biomaterial, the concentration of the polymers (e.g., PEGDA and gelatin or gelatin-based crosslinkable biomaterial) within the crosslinkable liquid composition, and the degree of functionalisation of the biomaterial.

The swelling ratio (SR) or degree of swelling (expressed in %) of a crosslinked composition at a predetermined time t may be defined as the ratio of the weight (Wₛ) of the crosslinked composition after immersion of the crosslinked composition for a predetermined time t in water or an aqueous composition minus the dry weight of the crosslinked composition at start (W_{d}) to the dry weight of the crosslinked composition at start (W_{d}), as calculated by the following formula (1):

$$SR ( \% ) = ( {W}_{s} - {W}_{d} ) / {W}_{d} \times 100 \qquad ( 1 )$$

In embodiments, the crosslinked composition may have a swelling ratio of 200% to 1000%, when fully hydrated. In embodiments, the crosslinked composition may have a swelling ratio of 300-800%, 400-800%, or 400-600%, when fully hydrated.

One of the main advantages of the uses or methods as taught herein over existing techniques is that it provides a long-term solution for treating a refractive error or a chronic or subacute corneal disease involving an irregularity of the cornea. Accordingly, in embodiments, the crosslinked composition may be stable at body temperature, such at about 37° C., preferably for a period of at least 1 month, at least 2 months, at least 3 months, at least 4 months, at least 5 months, at least 6 months, at least 1 year, at least 5 years, or at least 10 years.

Present inventors have found that by applying the crosslinkable liquid composition to the anterior corneal surface of the eye using a mold, the crosslinkable liquid composition and final crosslinked composition, can be precisely placed onto the anterior corneal surface of the eye while avoiding spilling of the crosslinkable liquid composition over the entire anterior eye surface and/or under the eyelids before crosslinking. Furthermore, the mold may already give a primary shape, diameter and/or preliminary curvature to the final corneal onlay that will be formed by the crosslinked composition. By creating the corneal onlay in situ, it perfectly fits the patient's corneal geometry, compared to a corneal onlay created separately.

In embodiments of the uses or methods as taught herein, the mold may be a corneal vacuum suction device, a corneal bath or a contact lens. In embodiments, the mold is a corneal vacuum suction device. In embodiments, the mold is an O-ring shaped corneal bath (also referred to as an o-ring). In alternative embodiments, the mold is a contact lens, preferably a contact lens, more preferably a scleral contact lens, that does not adhere to the biomaterial either in uncrosslinked or crosslinked state. In embodiments, the mold is a silicone hydrogel contact lens. A contact lens with a converging meniscus-shape allows shaping the crosslinked composition (i.e. biomaterial) into a lens-shape having a lens body (or lens (optical) zone) and a lens edge at the periphery of the lens body (or peripheral zone). Accordingly, the shaped crosslinked composition would use such a contact lens as a mold that is thinner towards its peripheral edges, and hence, typically having a peripherical edge which is less thick than when use is made of a corneal bath. As a result thereof, less or even no biomaterial will need to be removed upon correcting the curvature of the crosslinked composition, thereby making the uses or methods as taught herein more efficient and attractive.

In embodiments, the mold is a standard contact lens, which, the inventors have found, when used in the uses or methods as taught herein, inherently generates in an onlay for which the periphery is thicker than the center.

In embodiments, the shape of the mold may be adapted depending on the eye disorder to be treated. For example, for the treatment of farsightedness, the crosslinkable liquid composition can be added predominantly centrally of the cornea and for the treatment of nearsightedness, the crosslinkable liquid composition can be added predominantly peripherally of the cornea.

The shape of the mold may take into account the swelling of the crosslinkable liquid composition that may occur upon crosslinking and uptake of water by the biomaterial. For example, if a crosslinkable biomaterial is known to swell to twice its size upon crosslinking, and if a thickness of about 50.0 μm of the crosslinked composition (prior to eventual correcting the curvature) would be desired, the mold may be designed to only allow applying a layer of crosslinkable liquid composition with a thickness of about 25.0 μm.

In embodiments, the curvature of the contact lens may have a back central zone radius of from 8.0 to 15.0 mm, from 8.0 to 14.0 mm, from 8.0 to 13.0 mm, from 8.0 to 12.0 mm, from 8.0 to 11.0 mm, or from 8.0 to 10.0 mm.

Preferably, the curvature of the contact lens is so that it allows forming a void space or lens shaped cavity between the anterior surface of the cornea of the subject to be treated and the back surface of the central (optical) zone of the contact lens, while the back surface of the peripheral zone closely aligns with the peripheral zone of the cornea or sclera. This void space or lens shaped cavity can be filled with the crosslinkable liquid composition as described herein.

In embodiments, the shape of the mold is so that it allows the generation of a crosslinked composition or the subsequent shaping the crosslinked composition as to have an average thickness of from about 10.0 μm to about 400.0 μm, such as from about 20.0 μm to about 400.0 μm, from about 25.0 μm to about 400.0 μm, from about 50.0 μm to about 400.0 μm, from about 100.0 μm to about 400.0 μm, from about 200.0 μm to about 400.0 μm, or from about 100.0 μm to about 300.0 μm, prior to eventual correcting the curvature of the crosslinked composition.

In embodiments, the mold may cover at least 80%, at least 85%, at least 90%, or at least 95%, such as at least 95%; at least 96%, at least 97%, at least 98%, at least 99% or 100%, of the anterior corneal surface of the eye. In embodiments, the mold may completely cover the anterior corneal surface of the eye.

In embodiments, the shape of the mold may be such that it allows shaping the crosslinked composition as to have a diameter of from about 6.0 mm to about 9.0 mm, such as from about 6.0 mm to about 8.0 mm or from about 7.0 mm to about 9.0 mm. Present inventors realized that a wider diameter could risk covering the limbal epithelial cells, which differentiate and migrate to become corneal epithelial cells. Therefore, physically covering the limbus forms a risk of corneal epithelial cell ingrowth or impedes limbal stem cell differentiation, which is preferably avoided.

In embodiments, the mold, preferably the contact lens, has a total diameter (including the diameter of the central zone as well as peripheral zone of the mold) of from about 5.0 mm to about 30.0 mm, from 5.0 mm to 25.0 mm, from 10.0 mm to 25.0 mm, from 14.0 mm to 24.0 mm, from 5.0 mm to 10.0 mm, from 6.0 mm to 9.0 mm, or from 7.0 mm to 8.0 mm.

In embodiments, if UV irradiation or visible light is used to crosslink the crosslinkable liquid composition, the mold is capable of allowing the UV or visible light to reach the crosslinkable liquid composition. For example, if the mold is a contact lens, the contact lens allows passage of UV or visible light.

In embodiments, the method may comprise applying said mold onto the anterior corneal surface of the eye either before or after applying said crosslinkable liquid composition into said mold. In embodiments of the uses or methods as taught herein, the mold may be a corneal vacuum suction device, a corneal bath, or a contact lens and the method comprises applying said mold onto the anterior corneal surface of the eye either before or after applying said crosslinkable liquid composition into said mold.

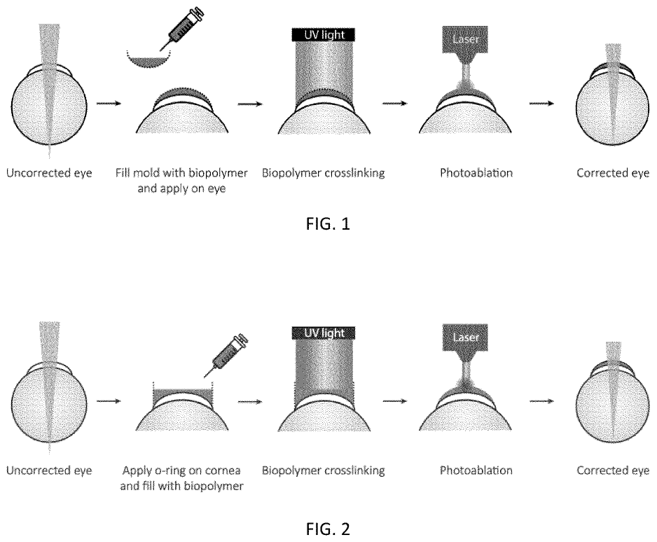

In embodiments, the mold such as a contact lens may be filled with the crosslinkable liquid composition prior to applying the mold and crosslinkable liquid composition to the anterior corneal surface of the eye, as illustrated in FIG. 1. In embodiments, the mold such as an O-ring shaped corneal bath (also referred to as an o-ring) may be applied to the anterior corneal surface of the eye prior to filling the mold with the crosslinkable liquid composition, as illustrated in FIG. 2.

A vacuum suction device acts similarly as an o-ring except that the suction device can be secured on the cornea and consists in different diameters to apply the onlay. In practice, a vacuum suction device (either a dedicated vacuum suction ring or a vacuum suction device from which the blade has been removed) may be vacuum locked on top of the eye, then the crosslinkable liquid composition as taught herein may be added and irradiated with UV or visible light. Then the vacuum suction device is taken off the eye. A vacuum suction device is for instance illustrated in FIG. 1B of Kim et al. (J. Vet Sci, 2015, 16, 349-356).

In embodiments, the crosslinkable liquid composition may be applied onto the anterior corneal surface of the eye in a volume from 10.0 to 200.0 μl, from 25.0 to 100.0 μl, preferably from 50.0 to 100.0 μl, such as about 50.0 μl. The combination of the mold and the limited amount of volume being used further allows avoiding spilling of the crosslinkable liquid composition over the entire anterior eye surface and/or under the eyelids before crosslinking.

In embodiments, the method may comprise maintaining the mold in place on the anterior corneal surface of the eye for the entire period of crosslinking the crosslinkable liquid composition.

In embodiments, if the mold is a contact lens, the center of the contact lens is placed onto the center of the anterior surface of the cornea.

In embodiments of the uses or methods as taught herein, the method may comprise removing the mold after crosslinking the crosslinkable liquid composition. In embodiments of the methods which involve a correction step, the method may comprise removing the mold after crosslinking the crosslinkable composition (and, where correction of the curvature of the crosslinked composition is envisaged, prior to correcting the curvature of the crosslinked composition).

In embodiments of the uses or methods as taught herein, the crosslinkable liquid composition may be applied as a single layer. In embodiments, the crosslinkable liquid composition may be provided as a single layer onto the anterior corneal surface of the eye. In line therewith, in embodiments, the crosslinked composition on the anterior corneal surface of the eye consists of a single layer of biomaterial.

In the methods envisaged herein, optionally, prior to applying the mold and the crosslinkable liquid composition onto the anterior corneal surface of the eye, the epithelial cells are removed from the cornea to expose the corneal stromal bed for grafting the corneal onlay thereon. The anterior corneal surface may be debrided of corneal epithelial cells by any means known in the art, such as by use of alcohol delamination, a blunt blade, a diamond burr, a cotton sponge or an Amoils brush. Alternatively the surgeon can opt to remove the epithelium after placing the mold (to only remove the epithelial cells in that area).

In embodiments, the uses and methods as taught herein may not comprise removal or damaging of the Bowman's layer, the corneal stroma, or a combination thereof prior to applying the crosslinkable liquid composition onto the anterior corneal surface of the eye.

In embodiments, the crosslinking may be performed by photocrosslinking, by exposure to O₂ or by one or more enzymes such as transglutaminases, transferases, tyrosinases and peroxidases. In case of enzymatic crosslinking, the enzyme and crosslinkable polymer can be mixed upon application in so-called dual barrel syringes which accommodate direct mixing in pre-defined ratios. In preferred embodiments, the crosslinking is performed by photocrosslinking, such as by use of UV light or visible light, more preferably by UV irradiation.

The term “photocrosslinking” as used herein refers to the process of using electromagnetic radiation, such as visible light or UV radiation, to crosslink compounds such as to crosslink the polymers of the crosslinkable liquid composition and/or to crosslink the polymers of the crosslinkable liquid composition with the anterior corneal surface. The electromagnetic radiation may be generated by a laser such as a pulsed laser.

In embodiments, photocrosslinking may be laser-assisted photocrosslinking.

The terms “radiation” and “electromagnetic radiation” may be used interchangeably herein.

In embodiments, the electromagnetic radiation is ultraviolet radiation or visible light.

In embodiments, the crosslinkable liquid composition is being crosslinked for a period of at least 1 minute. In embodiments, the crosslinkable liquid composition is being crosslinked for a period of at least 5 minutes, at least 10 minutes, at least 20 minutes, at least 30 minutes, or at least 40 minutes, preferably at least 30 minutes. In embodiments, the total dose of irradiation is at least 0.7 Joule (J)/cm², such as at least 0.8, at least 0.9, at least 1.0, at least 2.0, at least 3.0, at least 4.0 or at least 5.0 J/cm², preferably at least 1.0 J/cm² or at least 5 J/cm², such as about 5.4 J/cm². In embodiments, the power or intensity of irradiation ranges from 1 to 20 mW/cm², such as from 1 to 10 mW/cm².

The UV spectrum typically spreads from 250 to 450 nm. Preferably, UV irradiation has a wavelength of from 250 to 450 nm, from 300 to 450 nm or from 300 to 400 nm, such as about 365 nm.

In embodiments, the crosslinkable liquid composition is allowed to crosslink until at least 80.0%, preferably at least 90.0%, such as 99.9% or 100.0%, of the crosslinkable liquid composition is crosslinked. In embodiments, the crosslinkable liquid composition is being crosslinked using UV irradiation for a period of at least 1 minute. In embodiments, the crosslinkable liquid composition is being crosslinked using UV irradiation for a period of at least 5 minutes, at least 10 minutes, at least 20 minutes, at least 30 minutes, or at least 40 minutes, preferably at least 30 minutes.

In embodiments, the total dose of UV used during UV irradiation is at least 0.7 Joule (J)/cm², such as at least 0.8, at least 0.9, at least 1.0, at least 2.0, at least 3.0, at least 4.0 or at least 5.0 J/cm², preferably at least 1.0 J/cm² or at least 5 J/cm², such as about 5.4 J/cm². In embodiments, the power or intensity of irradiation ranges from 1 to 20 mW/cm². Preferably, the power or intensity of irradiation ranges from 1 to 10 mW/cm², which is inversely correlated to the irradiation time.

In the uses or methods as taught herein, the crosslinking is performed in situ, onto the anterior corneal surface of the eye. Hence, in the uses or methods as taught herein, the crosslinked composition is a corneal onlay. In embodiments, the crosslinking is performed at body temperature, such as at a temperature of from 35.0° C. to 40.0° C. or from 36.0° C. to 38.0° C.

The term “corneal onlay” generally refers to an optical device positioned between the epithelium and the stroma of the cornea to correct vision.

The use or method as taught herein provides a user-friendly and long-term treatment of the refractive error or the chronic or subacute corneal disease involving an irregularity of the cornea.

As the crosslinked composition on the anterior corneal surface of the eye is resistant to biodegradation, is less likely to result in post-procedure complications such as dry eye disease, pain or regression of the applied correction, and does not need to be replaced regularly.

In embodiments of the uses or methods as taught herein, the crosslinked composition or corneal onlay may be resistant to biodegradation for a period of at least 6 months, preferably at least 12 months. In embodiments, the crosslinked composition or corneal onlay may be resistant to degradation for a period of at least 6 months, preferably at least 12 months. In embodiments, the crosslinked composition or corneal onlay may be resistant to enzymatic degradation for a period of at least 6 months, preferably at least 12 months.

Enzymatic degradation may be simulated in vitro by incubating a crosslinked composition in a collagenase type I solution. In a degradation simulation experiment, a 0.1 Units (U) collagenase solution may be used, which is a 100 times overestimation of the collagenase concentrations in vivo. At start and at each time point, the weight of the crosslinked composition is recorded and divided by the initial weight to get the residual weight over time (expressed as a percentage). Collagenase solution is refreshed at every time point and samples are incubated e.g. at 37° C. (5% CO₂).

For crosslinked compositions at equilibrium, the residual weight (expressed in %) of a crosslinked composition at a predetermined time t may be defined as the ratio of the weight of the crosslinked composition after (enzymatic) degradation for a predetermined time t to the initial weight of the crosslinked composition. The (enzymatic) degradation or biodegradation (DR) (expressed in residual weight in %) of a crosslinked composition at a predetermined time t may be defined as the ratio of the hydrogel weight at time point (Wt) divided by the initial weight (W₀), i.e. the weight of the hydrogel when fully swollen to their equilibrium. (Wt/W₀)*100

In embodiments, the crosslinkable liquid composition or corneal onlay may be resistant to degradation by matrix metalloproteinases (MMPs) of the corneal epithelium, such as MMP-1, MMP-2, MMP-3, MMP-9, or a combination thereof.

Crosslinking of the crosslinkable liquid composition onto the anterior corneal surface of the eye leads to adherence between the crosslinked composition or corneal onlay and the anterior corneal surface of the eye through the formation of covalent bonds with the amino acids in the corneal collagen. In embodiments, the posterior surface of the crosslinked composition adheres to the Bowman's membrane of the eye with an adhesion strength of at least 10.0 kPa, and preferably with an adhesion strength from 10.0 to 100.0 kPa. The adhesion strength may be determined by any methods known in the art, such as by a lap shear test with a universal testing machine according to the ASTM F2255 using gelatin-coated glass slides.

In embodiments, the posterior surface of the crosslinked composition contacts the Bowman's membrane of the eye.

In embodiments, the crosslinked composition is flexible. The flexibility of the crosslinked composition may be altered by changing the concentration of the gelatin or gelatin-based crosslinkable biomaterial in the crosslinkable liquid composition, its degree of substitution, or its molecular weight. Preferably, the flexibility of the crosslinked composition is similar to native cornea.

In embodiments of the uses or methods as taught herein, the crosslinked composition or corneal onlay may have a diameter of from about 6.0 mm to about 9.0 mm and a thickness of from about 10.0 μm to about 400.0 μm prior to correcting the curvature of the crosslinked composition. For instance, the crosslinked composition may have a diameter of from about 6.0 mm to about 8.0 mm and a thickness of from about 10.0 μm to about 400.0 μm prior to correcting the curvature of the crosslinked composition, or the crosslinked composition may have a diameter of from about 7.0 mm to about 9.0 mm and a thickness of from about 10.0 μm to about 400.0 μm prior to correcting the curvature of the crosslinked composition.

In embodiments, the crosslinked composition may have a diameter of from about 6.0 mm to about 9.0 mm, such as from about 6.0 mm to about 8.0 mm or from about 7.0 mm to about 9.0 mm, prior to correcting the curvature of the crosslinked composition.

In embodiments, the crosslinked composition may have a thickness of from about 10.0 μm to about 400.0 μm, such as from about 20.0 μm to about 400.0 μm, from about 25.0 μm to about 400.0 μm, from about 50.0 μm to about 400.0 μm, from about 100.0 μm to about 400.0 μm, from about 200.0 μm to about 400.0 μm, or from about 100.0 μm to about 300.0 μm, prior to correcting the curvature of the crosslinked composition.

In embodiments, it can be of interest to correct the curvature of the onlay once the crosslinkable composition has been crosslinked onto the anterior corneal surface of the eye. More particularly, the crosslinked composition may be reshaped for optimal and/or patient-specific treatment of the eye disorder, such as a patient-tailored vision correction. For example, the radius of curvature of the crosslinked composition may be corrected, such as by use of photoablation, to treat the eye disorder. Reshaping of the crosslinked composition may change the refractive properties of the so-treated eye in a desired manner to correct the eye disorder, such as the refractive error.

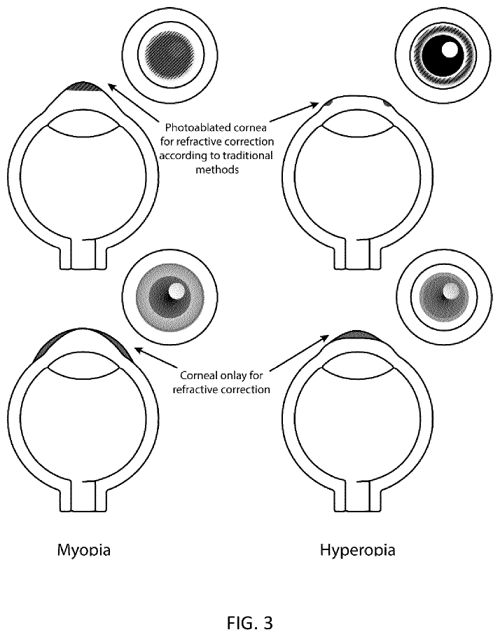

The person skilled in the art will understand that depending on the eye disorder to be treated, the radius of curvature of the crosslinked composition may be corrected differently. For example, for the treatment of farsightedness, biomaterial can be maintained predominantly centrally. Further for example, for the treatment of nearsightedness, biomaterial can be maintained predominantly peripherally. The ablation depth of the corneal onlay is correlated to the envisaged refractive correction and calculated similarly to current refractive laser surgery. Personalized treatment profiles, also known as nomograms, are generated and transferred to a laser for treatment. It is noted that the mode of refractive correction is different from current strategies because of the inherent nature of the corneal onlay technology. Whereas in case of myopia, laser refractive surgery subtracts peripheral tissue, a corneal onlay corrects myopia by adding peripheral tissue. Vice versa, laser refractive surgery corrects hyperopia by tissue dissection mid-peripherally, while a corneal onlay corrects this by adding tissue centrally (FIG. 3).

In embodiments, the crosslinked composition or corneal onlay may be photoablated to obtain a refractive correction, preferably a spherical refractive correction, in the range of from −20 diopters to +10 diopters. In embodiments, the crosslinked composition may be photoablated such that the crosslinked composition comprises at least a central portion having a substantially uniform thickness extending from the lower surface to the upper surface of the crosslinked composition such that the crosslinked composition has an optical power within a range from −20 diopters to about +10 diopters, preferably from −10 diopters to about +5 diopters, along at least the inner portion of the crosslinked composition. In particular embodiments, the refractive correction may also be obtained by shaping the curvature of the crosslinked material with a blade.

In embodiments, the crosslinked composition may be photoablated to obtain a non-spherical shape when the eye disorder is astigmatism.

In certain embodiments, particularly where the eye-disorder is not a refractive error but rather an irregularity of the cornea, the crosslinked composition may be adjusted to reproduce a natural surface, with minimal impact on vision.

In embodiments, the crosslinked composition may be photoablated to obtain a lens-shape, such as having a thickness of from 10.0 μm to 50.0 μm at the periphery of the outer portion of the crosslinked composition and extending to the central portion of the crosslinked composition with an increasing thickness to from 30.0 μm to 100.0 μm.

In embodiments, the upper surface of the crosslinked biomaterial may be photoablated to shape the upper surface.

Furthermore, the person skilled in the art will also understand that the possible deswelling of the crosslinked composition upon overgrowth of the epithelial cells should be taken into account when determining the amount of crosslinked composition that will be removed from the eye to treat the eye disorder.

Photoablation may be performed using a laser, such as an excimer laser.

In particular embodiments, the methods do not involve correction of the surface of the corneal onlay.

In embodiments, the correcting of the curvature of the crosslinked composition may not comprise removing corneal tissue, such as corneal stromal tissue, such as by photoablation.

In embodiments, no correction of the curvature of the crosslinked material is necessary. This can be the case where the layer of material is very thin and/or where a mold can be used which ensures exactly the desired shape and thickness of the material after crosslinking. In embodiments, photoablation of the corneal onlay may not be performed when the principal aim is to treat chronic or subacute corneal disease.

In embodiments, the method as taught herein is reversible, meaning that the crosslinked composition may be completely removed from the anterior corneal surface of the eye, if needed, such as by photoablation, hydrodissection, microkeratome or manual dissection.

After correcting the curvature of the crosslinked composition, the corneal epithelium may spontaneously reform originating from the corneal limbus, the corneal scleral transition zone.

Overgrow of the crosslinked composition by the corneal epithelium typically occurs within 1 to 2 weeks after correcting the curvature of the crosslinked composition. In embodiments, one or more therapeutic agents, such as NGF, may be administered to the eye to improve regrowth of the corneal epithelium.

The subject may post-operatively be treated with therapeutic agents that reduce pain and/or inflammation, such as corticosteroids and/or antibiotics.

The present application also provides aspects and embodiments as set forth in the following Statements:

Statement 1. A crosslinkable liquid composition for use in a method of treating an eye disorder in an eye of a subject, wherein the method comprises:

applying a crosslinkable liquid composition onto an anterior corneal surface of said eye comprising said eye disorder,
crosslinking the crosslinkable liquid composition on the anterior corneal surface of the eye, thereby obtaining a crosslinked composition on the anterior corneal surface of the eye, and optionally,
correcting the curvature of the crosslinked composition,
   wherein the method comprises introducing said crosslinkable liquid composition into a mold which is positioned onto the anterior corneal surface of the eye,
   wherein the eye disorder is a refractive error or a chronic or subacute corneal disease involving an irregularity of the cornea, and
   wherein the crosslinkable liquid composition comprises:
a polyethylene glycol diacrylate (PEGDA), and
a gelatin or a gelatin-based crosslinkable biomaterial.

Statement 2. The crosslinkable liquid composition for use according to statement 1, wherein the refractive error is selected from the group consisting of myopia, hyperopia, astigmatism and presbyopia.

Statement 3. The crosslinkable liquid composition for use according to statement 1 or 2, wherein the chronic or subacute corneal disease involving an irregularity of the cornea is selected from the group consisting of a corneal ulcer, a corneal erosion, a corneal ectasia, or a corneal irregularity caused by trauma or epithelial basement membrane dystrophy; preferably wherein the corneal ectasia is keratoconus.

Statement 4. The crosslinkable liquid composition for use according to any one of statements 1 to 3, wherein the gelatin-based crosslinkable biomaterial is selected from the group consisting of a gelatin methacrylate, a gelatin desaminotyrosine, a gelatin desaminotyrosyl tyrosine, a gelatin tyramine, and a thiolated gelatin.

Statement 5. The crosslinkable liquid composition for use according to any one of statements 1 to 4, wherein:
the crosslinkable liquid composition comprises from about 0.5% to about 15.0% (w/v) of the PEGDA;
the crosslinkable liquid composition comprises from about 5.0% to about 40.0% (w/v) of the gelatin or gelatin-based crosslinkable biomaterial;
the PEGDA has a molecular mass of about 250 g/mol to about 6000 g/mol; preferably of about 500 g/mol to about 1000 g/mol; and/or
the gelatin-based crosslinkable biomaterial is a functionalized gelatin having a degree of substitution of from about 40% to about 90%.

Statement 6. The crosslinkable liquid composition for use according to any one of statements 1 to 5, wherein the mold is a corneal vacuum suction device, a corneal bath or a contact lens.

Statement 7. The crosslinkable liquid composition for use according to any one of statements 1 to 6, wherein the method comprises applying said mold onto the anterior corneal surface of the eye either before or after applying said crosslinkable liquid composition into said mold.

Statement 8. The crosslinkable liquid composition for use according to any one of statements 1 to 7, wherein the method comprises removing the mold after crosslinking the crosslinkable composition.

Statement 9. The crosslinkable liquid composition for use according to any one of statements 1 to 8, wherein the crosslinkable liquid composition is applied as a single layer.

Statement 10. The crosslinkable liquid composition for use according to any one of statements 1 to 9, wherein the crosslinking is performed by photocrosslinking, by exposure to O₂ or by one or more enzymes; preferably wherein the crosslinking is performed by photocrosslinking such as by UV irradiation or irradiation with visible light.

Statement 11. The crosslinkable liquid composition for use according to any one of statements 1 to 10, wherein the crosslinkable liquid composition further comprises a photoinitiator and optionally a co-initiator.

Statement 12. The crosslinkable liquid composition for use according to any one of statements 1 to 11, wherein the crosslinkable liquid composition comprises a gelatin methacrylate, a polyethylene glycol diacrylate, and a photoinitiator; such as wherein the photoinitiator is Irgacure 2959 or Lithium phenyl-2,4,6-trimethylbenzoylphosphinate.

Statement 13. The crosslinkable liquid composition for use according to any one of statements 1 to 12, wherein the crosslinkable liquid composition comprises a gelatin desaminotyrosine, a polyethylene glycol diacrylate, a photoinitiator, and optionally a co-initiator; such as wherein the photoinitiator comprises tris(2,2′-bipyridine) ruthenium (II) or riboflavin and the co-initiator is sodium persulphate.

Statement 14. The crosslinkable liquid composition for use according to any one of statements 1 to 13, wherein the crosslinked composition has a diameter of from about 6.0 mm to about 9.0 mm and a thickness of from about 10.0 μm to about 400.0 μm, prior to correcting the curvature of the crosslinked composition.

Statement 15. The crosslinkable liquid composition for use according to any one of statements 1 to 14, wherein the crosslinked composition is resistant to biodegradation for a period of at least 6 months, preferably at least 12 months.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and scope of the appended claims.

The herein disclosed aspects and embodiments of the invention are further supported by the following non-limiting examples.

### EXAMPLES

### Example 1: Swelling Degree of a Crosslinked Composition Obtained from a Crosslinkable Liquid Composition for Use in a Method of Treating an Eye Disorder in an Eye of a Subject According to Embodiments of the Invention

The materials used are as follows: Eppendorf tubes amber colored (VWR, 525-1223); Poly ethylene glycol diacrylate (Merck, 907227-1G); Lithium phenyl-2,4,6-trimethylbenzoylphosphinate (Merck, 900889-1G); Gelatin methacrylate DS90 or DS160 (Rousselot); Phosphate buffered saline 1× (Thermo Fisher, 14200083); CCL-365 UV lamp (Vario); UV crosslinker CL-3000 (AnalytikJena); Hydrogel formulations were prepared in eppendorf tubes according to the desired concentrations of the gelatin or gelatin-based crosslinkable biomaterial, photoinitiator, and PEGDA; the crosslinkable biomaterial was then completely dissolved by mixing and vortexing. Droplets of 50 μL of the crosslinkable liquid composition were pipeted onto parafilm and irradiated with CCL-365 UV lamp for 5 minutes at 18 mW/cm² or with a UV crosslinker (AnalytikJena) at 1 mW/cm² to obtain an accumulated UV dosage of 5.4 J/cm².

To further analyse the properties of the compositions, the crosslinked compositions were then dried at 50° C. for 2 hours. The weight at time point zero (W_{d}) was recorded. They were then placed in PBS at 37° C. and the swollen crosslinked compositions were weighed at regular time intervals (Wₛ). Before weighing, surface water was removed with a tissue. The swelling rate at each time point was calculated according to the following equation:

$$SR ( \% ) = ( {W}_{s} - {W}_{d} ) / {W}_{d} \times 100$$

First, the swelling and degradation behaviour of a corneal onlay was tested by adding a synthetic polymer (poly ethylene glycol diacrylate PEGDA; Mn=4,000 Da) to a gelatin-based crosslinkable material (gelatin methacrylate GelMa; Mn=90 kDa or 160 kDa) to have more control of the rate and degree of swelling, and to protect to corneal onlay to acute and chronic degradation. Several concentrations (0%, 1%, or 2% w/v PEGDA) and molecular weights of the PEGDA (90 kDa or 160 kDa) were tested. Several concentrations (10% or 20% w/v) of GelMA (Rousselot) were tested. The solvent of the crosslinking compositions was in all cases 1×PBS, while the photoinitiator concentration (LAP) is 0.125%.

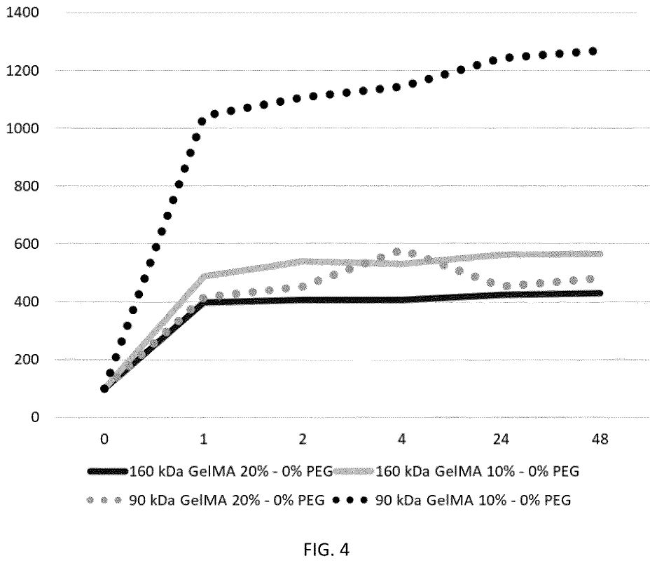

The crosslinked compositions were prepared as described above. After crosslinking of the comparative gelatin-based crosslinkable material, it imbibed multiple times its own weight in water to reach a final thickness. Comparative crosslinked compositions made up of 10% versus 20% gelatin-based crosslinkable material (GelMA) displayed a different swelling behaviour with gelatin-based crosslinkable materials in low concentrations swelling up more than more concentrated hydrogels (FIG. 4). Furthermore, comparative gelatin-based crosslinkable materials with a higher molecular weight (160 kDa) swelled less than their lower molecular weight counterpart (90 kDa) (FIG. 4). The rate of swelling until its equilibrium was not different in hydrogels when changing the molecular weight of gelatin (FIG. 4).

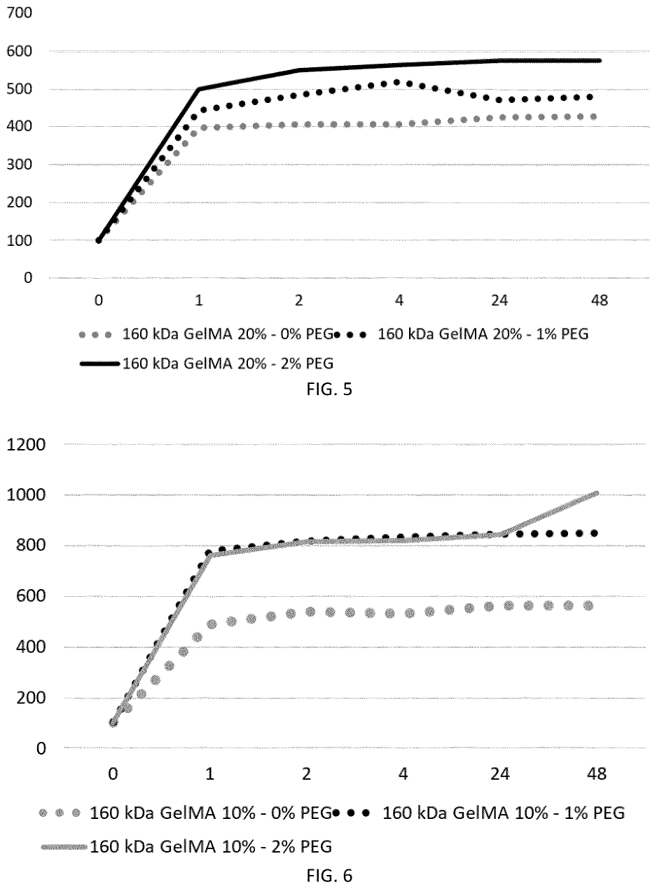

Crosslinked compositions illustrating the invention that were made with an additional percentage of PEGDA, took up more water over time (FIG. 5 and FIG. 6). For example, as shown in FIG. 5, the highest swelling rate was obtained for the crosslinked composition illustrating the invention comprising 20% GelMA (160 kDa) with 2% PEGDA, followed by 20% GelMA (160 kDa) with 1% PEGDA. The lowest swelling rate was obtained for the crosslinked composition comprising 20% GelMA but no PEGDA. (FIG. 5). Further, crosslinked composition comprising 10% GelMA (160 kDa) with 2% PEGDA had the highest swelling rate which was similar to 10% GelMA (160 kDa) with 1% PEGDA. The lowest swelling rate was obtained for the crosslinked composition comprising 10% GelMA but no PEGDA. (FIG. 6). The rate of swelling until its equilibrium was not different in hydrogels with or without PEGDA and was reached between 2-6 hours after placing them in phosphate buffered saline (FIG. 5 and FIG. 6).

### Example 2: Resistance to Enzymatic Degradation of a Crosslinked Composition Obtained from a Crosslinkable Liquid Composition for Use in a Method of Treating an Eye Disorder in an Eye of a Subject According to Embodiments of the Invention

The materials used were as follows: Eppendorf tubes amber colored (VWR, 525-1223); Poly ethylene glycol diacrylate (Merck, 907227-1G); Lithium phenyl-2,4,6-trimethylbenzoylphosphinate (Merck, 900889-1G); Gelatin methacrylate DS90 or DS160 (Rousselot); Collagenase (Merck, C9891-500 MG); Phosphate buffered saline 1×(Thermo Fisher, 14200083); CCL-365 UV lamp (Vario) UV crosslinker CL-3000 (AnalytikJena).

First, crosslinkable liquid compositions were prepared in eppendorf tubes according to the desired concentrations of functionalized gelatin, photoinitiator, and PEGDA. The crosslinkable biomaterial was completely dissolved by mixing and vortexing. Droplets of 50 μl of crosslinkable liquid composition were pipeted onto parafilm and these were irradiated with CCL-365 UV lamp for 5 minutes at 18 mW/cm2 or with a UV crosslinker (AnalytikJena) at 1 mW/cm2 to obtain an accumulated UV dosage of 5.4 J/cm2.

The crosslinked compositions were then incubated in PBS until they reach final thickness (>24 hours) and weigh the hydrogels (W0).

The crosslinked compositions were then placed in collagenase solution (0.1 U) and weighed at regular time intervals (Wt). Before weighing, surface water was removed with a tissue. The collagenase solution was refreshed after every measurement with a fresh made collagenase solution.

Enzymatic degradation was determined by calculating the residual weight according to the following equation: residual weight=(Wt/W0)*100.

Since the function of the corneal onlay is refractive correction due to its geometric shape (i.e., refractive power), it should not degrade over time. Two different pathways for possible degradation based on the time of onset of degradation were identified and defined: acute and chronic. The first period after epithelial debridement is a short window of time (7-14 days) where the material is exposed and corneal epithelial cells still need to grow over the onlay. This is the moment the eyelid friction and matrix metalloproteases (MMPs) from the tear fluid come into direct contact with the onlay. Once the cells form a new confluent corneal epithelium, they form a protective barrier to the eyelids and tear fluid MMP as this is one of their primary functions in normal corneas. Chronic degradation (or tissue remodelling) is also possible, though this would occur under intact corneal epithelial layers more slowly.

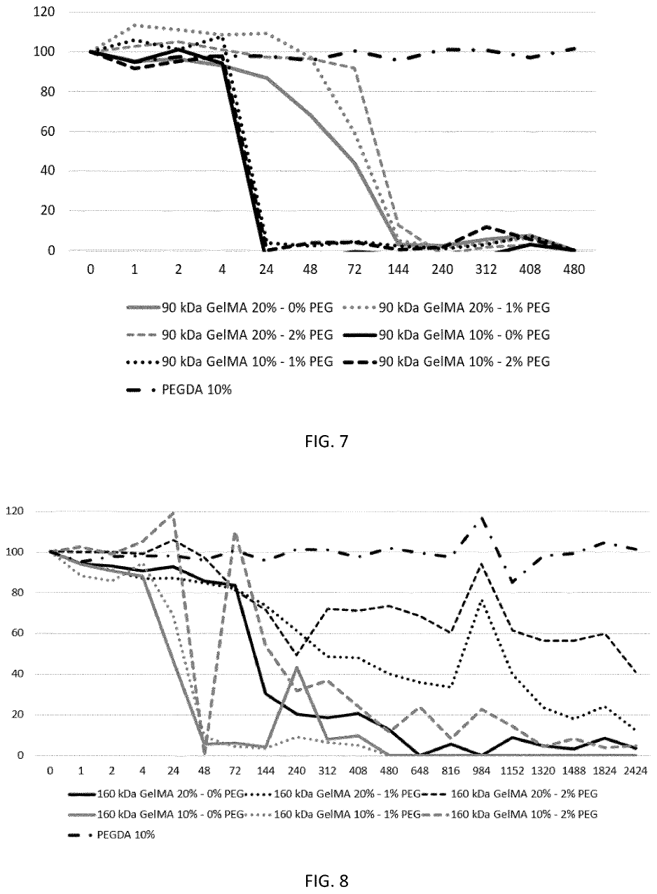

As shown in FIG. 7, comparative compositions were prepared as follows: 10% or 20% gelatin methacrylate (GelMa) Mn=90 kDa, or 10% poly ethylene glycol diacrylate (PEGDA) Mn=4,000 Da. Crosslinked compositions according to an embodiment of the invention were prepared as follows: 10% or 20% 90 kDa GelMA supplemented with 1% or 2% of PEGDA (shown in FIG. 7). The solvent of the crosslinking compositions was in all cases 1×PBS, while the photoinitiator concentration (LAP) is 0.125%.

As shown in FIG. 8, comparative compositions were prepared as follows: 10% or 20% 160 kDa GelMA, or 10% PEGDA. Crosslinked compositions according to an embodiment of the invention were prepared as follows: 10% or 20% 160 kDa GelMA supplemented with 1% and 2% of PEGDA. Enzymatic degradation was simulated in vitro by incubating hydrogels in a collagenase type I solution. In this degradation simulation experiment, a 0.1 Units (U) collagenase solution was used, which is a 100× overestimation of the collagenase concentrations in vivo. At each time point t, the weight of the crosslinked compositions was recorded (Wt) and divided by the initial weight (W₀) to get the residual weight over time. Collagenase solution was refreshed on every time point and samples were incubated at 37° C. (5% CO2). The kinetic course of the enzymatic degradation is displayed in FIG. 7 (90 kDa GelMA) and FIG. 8 (160 kDa GelMA) where the residual weight over time can be followed.

Table 1 serves as a summary to highlight the timepoints at which full degradation of the hydrogels was observed. When looking to both molecular weight (MW) gelatin variants, the time to full degradation was already significantly delayed by increasing the gelatin concentration. The resistance to degradation was further increased by adding PEGDA from 1-2%. When adding 2% PEGDA to 90 kDa GelMA 10% the time to degradation doubled from one day to two days. When looking to the 20% hydrogels of the same 90 kDa hydrogels, the degradation time increased from 6 to 10 days. In the higher MW gelatin subtypes, adding 2% PEGDA to 10% GelMA increased the time to degradation from 2 to 10 days. When looking to the 20% GelMA 160 kDa, the hydrogels were not even fully degraded after 101 days when adding 1-2% PEGDA. The positive control of 10% PEGDA did not dissolve over the course of the experiment (FIGS. 7 and 8).

TABLE 1
The time to full degradation of crosslinked compositions obtained
from crosslinkable liquid compositions according to embodiments
of the invention and from comparative compositions

| | Time to | | | Time to | |
|---|---|---|---|---|---|
| Composition | degradation | | Composition | degradation | |
| (90 kDa MW) | (days) | | (160 kDa MW) | (days) | |
| GelMA 10% - 0% PEG | 1 | day | GelMA 10% - 0% PEG | 2 | days |
| GelMA 10% - 1% PEG | 1 | day | GelMA 10% - 1% PEG | 2 | days |
| GelMA 10% - 2% PEG | 2 | days | GelMA 10% - 2% PEG | 10 | days |
| GelMA 20% - 0% PEG | 6 | days | GelMA 20% - 0% PEG | 6 | days |

| | | | | |
|---|---|---|---|---|
| GelMA 20% - 1% PEG | 6 | days | GelMA 20% - 1% PEG | 88% degraded after |
| | | | | 101 days |
| GelMA 20% - 2% P | 10 | days | GelMA 20% - 2% P | 60% degraded after |
| | | | | 101 days |

| | | | |
|---|---|---|---|
| PEGDA 10% | Not degraded | PEGDA 10% | Not degraded |

### Example 3: Exemplary Method of Treating Farsightedness with the Compositions of the Invention (Such as in FIGS. 1 and 2)

The refractive error of the patient has been diagnosed according to an established method.

After preparing the patient for the procedure, the patient's corneal epithelium is removed using diluted alcohol.

The ring of a vacuum suction device is placed on top of the cornea of the patient. About 50 μl of the uncrosslinked polymer (dissolved photocrosslinkable polymer comprising a polyethylene glycol diacrylate (PEGDA), and a gelatin or a gelatin-based crosslinkable biomaterial with photoinitiator Irgacure 2595)) is applied in the vacuum suction device.

The treated eye is irradiated to fully crosslink and adhere the corneal onlay with about e.g. 5.4 J/cm² of ultraviolet light of 365 nm. After the crosslinking the ring of the suction device is removed.

The polymer covalently adheres to extracellular matrix of the eye during the crosslinking process and is allowed to rehydrate (isotonic eye drops can be administered).

The crosslinked material is allowed to equilibrate for a period of about 4 hours.

Laser refractive surgery is then performed on the corneal onlay (and not the cornea tissue) thereby correcting the farsightedness.

### Example 4: In Vivo Experiments in Rats and Rabbits to Prepare a Crosslinked Composition Obtained from a Crosslinkable Liquid Composition for Use in a Method of Treating a Refractive Error According to Embodiments of the Invention

To test the feasibility of treating a refractive error of the eye of a subject using a crosslinkable liquid composition according to an embodiment of the invention, in vivo experiments were performed in rats (n=10) and rabbits (n=4).

### Animal Experiments in Rats

### Protocol

Anesthesia was performed as follows: induction: 4.5% Isoflurane, maintenance: 1-2%, and topical application of anesthesia oxybrucain (0.4%).

Surgical preparation was performed as follows: The third eyelid was cut away with microscissors or was sutured to the upper eye lid. Debridement of epithelium was performed with 20% ethanol for 10 seconds and manual scraping of epithelial cells. The anterior surface was washed with phosphate-buffered saline and dried.

Onlay procedure was performed by applying 1 μl of onlay solution (15% (w/v) GelMA/1% (w/v) PEGDA/0.0625% (w/v) LAP). Crosslink was performed with UV light (365 nm) for 5 min, 18 mW/cm² with Vario CL-365 crosslinking apparatus.

The onlay was validated by imaging with an intraoperative optical coherence tomography (OCT) apparatus (iVue).

### Results

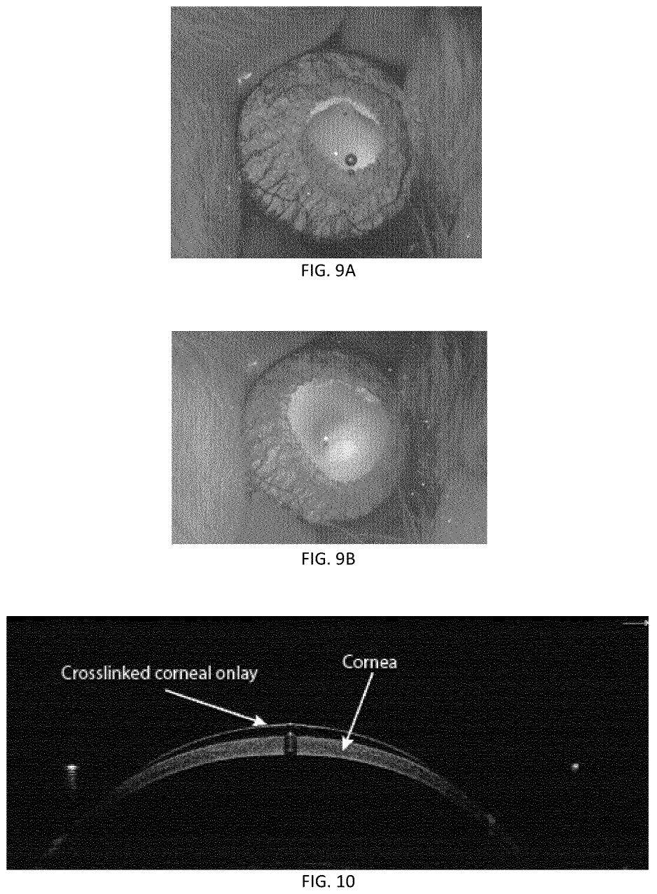

FIG. 9 illustrates a rat's eye with the crosslinkable liquid composition (1 μl) before crosslinking (FIG. 9A) and with the corneal onlay after crosslinking of the crosslinkable liquid composition (FIG. 9B). The OCT image demonstrated the presence of the crosslinked corneal onlay on the cornea of the rat's eye (FIG. 10). Given that a crosslinked corneal onlay changes the curvature of the cornea, the results confirm the feasibility of treating a refractive disorder using a crosslinkable liquid composition in accordance with the uses or methods illustrating the invention. The skilled person understand that the curvature of the crosslinked corneal onlay is then to be adjusted depending on the refractive error to be treated.

### Animal Experiments in Rabbits

### Protocol

Anesthesia was performed as follows: induction: ketamine (1 mL/kg) and Medetomidine (0.2 mL/kg), maintenance: 3% isoflurane, and topical application of anesthesia oxybrucain (0.4%).

Surgical preparation was performed as follows: Epithelial debridement with 20% ethanol for 10 seconds and manual scraping of epithelial cells. The anterior surface was wash with phosphate-buffered saline and dried.

Onlay procedure was performed by apply 10 μl of onlay solution (15% (w/v) GelMA/1% (w/v) PEGDA/0.0625% (w/v) LAP). Crosslink was performed with UV light (365 nm) for 5 min, 18 mW/cm² with Vario CL-365 crosslinking apparatus. The onlay was covered with a soft contact lens to protect the corneal onlay against grooming of the rabbit.

The onlay was validated by imaging with intraoperative OCT (iVue).

### Results

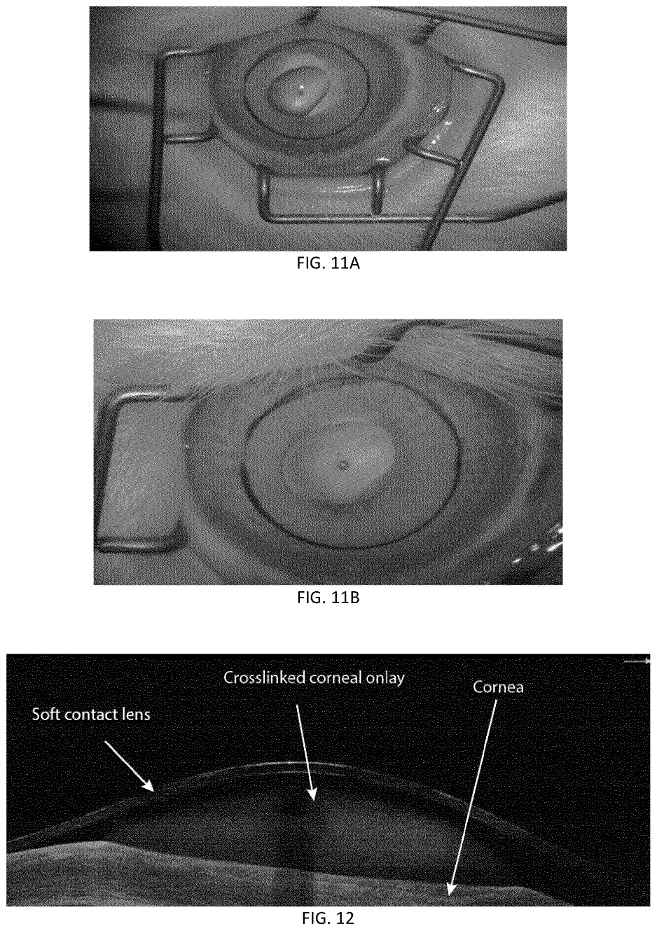

FIG. 11 shows a rabbit's eye with the crosslinkable liquid composition (10 μl) before crosslinking (FIG. 11A) and with the corneal onlay after crosslinking of the crosslinkable liquid composition (FIG. 11B). The OCT image demonstrated the presence of the crosslinked corneal onlay on the cornea of the rabbit's eye (FIG. 12). The soft contact lens is also visible (FIG. 12).

Given that a crosslinked corneal onlay alters the curvature of the cornea, the results confirm the feasibility of treating a refractive error using a crosslinkable liquid composition in accordance with the uses or methods illustrating the invention. The skilled person understands that the curvature of the crosslinked composition is then to be corrected, e.g. by a laser, depending on the refractive error to be treated.

1.-12. (canceled)

13. A method of treating an eye disorder in an eye of a subject in need thereof, the method comprising the steps of:
applying a crosslinkable liquid composition onto an anterior corneal surface of said eye comprising said eye disorder,
crosslinking the crosslinkable liquid composition on the anterior corneal surface of the eye, thereby obtaining a crosslinked composition on the anterior corneal surface of the eye, and,
correcting the curvature of the crosslinked composition,
wherein the method comprises introducing said crosslinkable liquid composition into a mold which is positioned onto the anterior corneal surface of the eye,
wherein the eye disorder is a refractive error selected from myopia, hyperopia, astigmatism and presbyopia, and
wherein the crosslinkable liquid composition comprises:
a polyethylene glycol diacrylate (PEGDA),
a gelatin or a gelatin-based crosslinkable biomaterial, and
a photoinitiator and optionally a co-initiator.

14. The method according to claim 13, wherein the gelatin-based crosslinkable biomaterial is selected from a gelatin methacrylate, a gelatin desaminotyrosine, a gelatin desaminotyrosyl tyrosine, a gelatin tyramine, and a thiolated gelatin.

15. The method according to claim 13, wherein:
the crosslinkable liquid composition comprises from 0.5% to 15.0% (w/v) of the PEGDA;
the crosslinkable liquid composition comprises from 5.0% to 40.0% (w/v) of the gelatin or gelatin-based crosslinkable biomaterial;
the PEGDA has a molecular mass of 250 g/mol to 6000 g/mol;
the gelatin-based crosslinkable biomaterial is a functionalized gelatin having a degree of substitution of from 40% to 90%; or
any combination thereof.

16. The method according to claim 13, wherein the PEGDA has a molecular mass of 500 g/mol to 1000 g/mol.

17. The method according to claim 13, wherein the mold is a corneal vacuum suction device, a corneal bath or a contact lens.

18. The method according to claim 13, wherein the method comprises applying said mold onto the anterior corneal surface of the eye either before or after applying said crosslinkable liquid composition into said mold.

19. The method according to claim 13, wherein the method comprises removing the mold after crosslinking the crosslinkable composition.

20. The method according to claim 13, wherein the crosslinkable liquid composition is applied as a single layer.

21. The method according to claim 13, wherein the crosslinking is performed by photocrosslinking.

22. The method according to claim 21, wherein the crosslinking is performed by photocrosslinking by UV irradiation or irradiation with visible light.

23. The method according to claim 13, wherein the crosslinkable liquid composition comprises a gelatin methacrylate, a polyethylene glycol diacrylate, and a photoinitiator.

24. The method according to claim 23, wherein the photoinitiator is Irgacure 2959 or Lithium phenyl-2,4,6-trimethylbenzoylphosphinate.

25. The method according to claim 13, wherein the crosslinkable liquid composition comprises a gelatin desaminotyrosine, a polyethylene glycol diacrylate, a photoinitiator, and optionally a co-initiator.

26. The method according to claim 25, wherein the photoinitiator comprises tris(2,2′-bipyridine) ruthenium (II) or riboflavin and the co-initiator is sodium persulphate.

27. The method according to claim 13, wherein the crosslinked composition has a diameter of from 6.0 mm to 9.0 mm and a thickness of from 10.0 μm to 400.0 μm, prior to correcting the curvature of the crosslinked composition.

28. The method according to claim 13, wherein the crosslinked composition is resistant to biodegradation for a period of at least 6 months.

29. The method according to claim 28, wherein the crosslinked composition is resistant to biodegradation for a period of at least 12 months.
